(19) United States
(12) Patent Application Publication
(10) Pub. No.: US 2008/0214387 A1
(43) Pub. Date: Sep. 4, 2008

(54) Process For Modifying Catalysts and the Use of the Catalysts

(76) Inventors: Daniel Ostgard, Kleinostheim (DE); Virginie Duprez, Toulon (FR); Roberta Olindo, Munchen (DE); Stefan Roder, Sinntal (DE); Monika Berweiler, Maintal (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 11/719,148

(22) PCT Filed: Nov. 12, 2004
(86) PCT No.: PCT/EP2004/012862

§ 371 Date: Jan. 28, 2008

(51) Int. Cl. B01J 23/02; C07C 59/10; C07C 211/02; B01J 23/10; B01J 23/28; B01J 23/44; B01J 23/72; B01J 23/75; C07C 291/06; B01J 23/755; B01J 23/745; B01J 23/46; B01J 23/42; B01J 23/26; B01J 31/02; B01J 23/04

(52) U.S. Cl. 502/165; 562/587; 564/463; 502/337; 502/331; 502/345; 502/325; 502/167; 502/312; 502/315; 502/316; 502/318; 502/328; 502/330; 502/302; 502/340; 502/332; 502/335; 502/341; 502/306; 502/308; 502/311; 502/314; 502/313; 502/326; 502/327; 502/338; 502/339; 502/317; 502/346; 558/299; 502/246

(57) ABSTRACT

Process for modifying catalysts via the deposition of carbon containing residues in the presence of one or more solvents, where the gas phase over the catalyst treatment solution during the treatment is air or an inert gas, and/or the liquid phase contains a templating agent and/or base. The modified catalyst can be used for stereo-, chemo- and regioselective transformations of organic compounds.

The subject of the invention is a process for modifying catalysts and the use of the catalysts.

Stereoselectivity involves transforming an organic molecule into an optically active one due to the introduction of one or more chiral centers into the molecule.

An example of a stereoselective reaction would be the controlled addition of two hydrogen atoms to one face or the other of an unsaturated prochiral compound such as an olefin, imine, carbonyl, carboxylic acid, ester, anhydride, oxime, sulfoxide or any other prochiral organic double bound for the creation of at least one chiral center.

For example, the hydrogenation of fructose could either generate mannitol or sorbitol depending on which species from the mutarotation of this ketose is preferentially adsorbed on the surface and hydrogenated.

Chemoselectivity involves the preferred transformation of one type of moeity over one or more others. This chemoselective preference may be due to the stronger adsorption of one moeity over the others, the reduced steric hindrance of the preferred functional group over the others, the preferred coordination of the transformed functional group to promoters or other differentiating factors.

Regioselectivity deals with the preferred transformation of a functional group based on its location in the molecule. In such cases, it is usually the functional groups in the least sterically hindered locations that are preferentially converted into the desired functionality. Factors such as steric, inductive and resonance effects of neighboring moeities will influence the regioselectivity of a molecule's transformation.

Additionally, other factors not listed here may also decide the regioselectivity of a reaction.

In the case of regioselective reactions, the functional groups that compete for the same reaction may or may not be the same and the only criteria that decides which moeity reacts is the location of that moeity in the molecule.

Examples of reactions that may be considered both chemoselective as well as regioselective would be the hydrogenation of citral (and other unsaturated aldehydes) to either the unsaturated alcohol or the saturated aldehyde, and the selective hydrogenation of unsaturated fatty nitrites to either unsaturated fatty amines or saturated nitrites.

A pure regioselective example would be the selective hydrogenation of a terminal carbon double bond in the presence of a di-, tri- or tetrasubstituted one(s) in the same molecule. Of course, the hydrogenation of the most substituted olefin over the least substituted would also be an example, albeit very rare, of a regioselective reaction.

Degischer et al. U.S. Pat. No. 6,521,564 also found that treating an activated Ni catalyst with formaldehyde can have a beneficial effect on its primary amine formation during nitrile hydrogenation.

However they did not utilize catalyst promotion with other elements, the use of fixed bed forms and their treatment procedure left high ppm levels of Ni in the treatment solution leading to undesired effects and additional costs through the disposal and/or treatment of such a waste stream. Moreover Degischer et al. performed their treatment only under inert gases and in the absence of molecular oxygen, and our results showed that the best results can also be achieved without inert gases.

According to the invention we have found, that oxidation of the catalytic surface does occur during its treatment with formaldehyde, sodium formate or any other similar derivatives. Thus the use of expensive inert gases is not needed and the additional oxygen may actually provide a benefit. One part of this invention deals with performing this modification procedure without the problems involved with the dissolved Ni in the treatment solution and/or the use of inert gases.

An additional part of this invention involves the use of the promoted catalyst for this modification and this catalyst may optionally be made and used as a fixed bed catalyst.

The present invention relates to the use of modified activated base metal catalyst for the improved selectivity of organic reactions.

The improvements according to the invention in selectivity involved enhanced stereo-, chemo- and regioselective transformations and these enhancements were carried out by the deposition of carbon containing residues on the Raney-type base metal catalyst. These carbon containing residues may also contain oxygen, sulfur, nitrogen, hydrogen and other atoms that are commonly present in organic molecules.

The preferred precursors for the deposition of carbon residues include formaldehyde and metal salt formates. However, other molecules such as carbon monoxide, carbon dioxide, aldehydes, ketones, amides (such as formamide), carboxylic acids, salts of carboxylic acids and other organic molecules that interact strongly with activated metal surfaces can also be used.

The modification method of this invention can also be used in coordination with other strongly adsorbing molecules, that may act as templates for controlling the structure of the carbonaceous deposits.

The subject of the invention is a process for modifying catalysts, where Ni, Cu, Co and mixtures thereof catalysts are modified via the deposition of carbon containing residues with materials such as formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in the liquid phase in the presence of one or more solvents at temperatures ranging from above 0 to 150° C. for a period of time ranging from instant contact to longer than 24 hours, when the catalyst is to be stored in the treatment solution, characterized in that the gas phase over the catalyst treatment solution during the treatment is air and no precautions were taken to remove the dissolve oxygen from the treatment solution,
or the gas phase over the catalyst during the treatment is an inert gas or air, when the catalysts are Cu catalysts,
or the gas phase over the catalyst treatment solution during the treatment is an inert gas or air, when the catalysts are a Ni, Cu, Co and mixtures thereof catalysts and the catalysts are doped or promoted with one or more elements,
or the gas phase over the catalyst treatment solution during the treatment is an inert gas or air, when the catalysts are a Ni, Cu, Co and mixtures thereof catalysts and the catalysts are optionally doped or promoted with one or more elements and the liquid phase contains a templating agent,
or the gas phase over the catalyst treatment solution during the treatment is an inert gas or air, when the catalyst are a Ni, Cu, Co and mixtures thereof catalysts and the catalysts are optionally doped or promoted with one or more elements and/or the liquid phase contains a templating agent and/or one or more bases before, during and/or after addition of the modifier to the modification solution,
or the gas phase over the catalyst treatment solution during the treatment is an inert gas or air, when the catalysts are Ni, Cu, Co and mixtures thereof catalysts and the catalysts are optionally doped or promoted with one or more elements and the liquid phase contains one or more bases before, during and/or after the addition of the modifier to the modification solution,
or the treatment of fixed bed catalysts, when the catalysts are Ni, Cu, Co and mixtures thereof catalysts and the catalysts are optionally doped or promoted with one or more elements and the process is protected by an inert gas and/or a liquid phase present as either a thin film on the catalyst or a solution,
or the treatment of fixed bed catalysts, when the catalysts are Ni, Cu, Co and mixtures thereof catalysts and the catalysts are optionally doped or promoted with one or more elements and/or in the presence of one or more bases and/or in the presence of a template where the process is protected by an inert gas and/or a liquid phase present as either a thin film on the catalyst or as a solution,
or the treatment of Ni, Cu, Co and mixtures thereof catalysts and the catalysts are optionally doped or promoted with one or more elements and optionally the modification medium contains a template and optionally the modification takes place in the presence of one or more bases, where the final modified catalyst is embedded in a fatty amine,
or the treatment of Ni, Cu, Co and mixtures thereof catalysts and the catalysts are optionally doped or promoted with one or more elements and optionally the modification medium contains a template and optionally the modification takes place in the presence of one or more bases, where the final catalyst is embedded in a primary fatty amine.

When the modification agent is formaldehyde, it can also be used in the form of metaldehyde or paraformaldehyde. Preferably, formaldehyde is used in the form of its aqueous solution, i.e. as formalin, in which case water then forms at least part of the liquid dispersion medium.

The “lower aliphatic aldehyde” which can be used as the modification agent in the present invention is preferably an aldehyde of the formula R¹CHO in which R¹ is an alkyl group with 1 to 5 carbon atoms optionally substituted with hydroxy. The alkyl group can be straight-chained or branched depending on the number of carbon atoms. In the case where the alkyl group is substituted with hydroxy, one or more hydroxy substituents can be present. Preferably, acetaldehyde is used as such a modification agent.

The “aromatic aldehyde” which can be used as the modification agent in the present invention is especially an aldehyde of the formula R²CHO in which R² is an aryl or heteroaryl group. The term “aryl” as used herein embraces not only the usual unsubstituted aryl groups, i.e. phenyl and naphthyl, but also the corresponding substituted phenyl and naphthyl, but also the corresponding substituted phenyl and naphthyl groups. The substituents may be, for example, halogen atoms and C₁₋₄-alkyl, hydroxy, C₁₋₄-alkoxy, amino, carbamoyl, and phenyl groups. In each case one or more substituents can be present.

Fluorine, chlorine, bromine, or iodine is to be understood under the term “halogen”. An alkyl or alkoxy group can be straight chain or branched depending on the number of carbon atoms. In the case of multiple substituents the substituents may be the same or different. Normally, not more than 5 (for phenyl) or 7 (for naphthyl) halogen atoms, 3 alkyl groups, 2 hydroxy groups, 3 alkoxy groups, 2 amino groups, 2 carbamoyl groups, or one phenyl group may be present as a substituent.

The term “heteroaryl” as used herein embraces heteroaryl groups, which have one or more hetero atoms in the ring, such as nitrogen, oxygen, and/or sulphur atoms. Pyridyl and pyrimidinyl are examples of such heteroaryl groups. Preferably, benzaldehyde or anisaldehyde is used as the aromatic aldehyde in the role of the modification agent.

The “aliphatic, aromatic or mixed aliphatic/aromatic ketone”, which can be used as the modification agent in the present invention is preferably a ketone of the formula R³COR⁴ in which R³ and R⁴ each independently signify an alkyl, aryl, or heteroaryl group. The terms ,alkyl, aryl” or ,heteroaryl” as used herein are to be understood as above in connection with the definitions or R¹ and R². Preferably, acetone is used as such a modification agent.

In one embodiment of the invention carbon monoxide, formaldehyde or a lower aliphatic aldehyde is used as the modification agent. In another embodiment of the invention carbon dioxide, metal formates (e.g., sodium formate) or a lower aliphatic metal carboxylate is used as the modification agent.

According to the invention the process for modifying Ni, Cu, Co and mixtures thereof catalysts, can be characterized in treating the catalysts via the deposition of carbon containing residues with materials such as formaldehyde, metal salt formates, carbon monoxide, carbon dioxide, aldehydes, ketones, amides (e.g., formamide), carboxylic acids, salts of carboxylic acids and organic molecules that interact strongly with metal surfaces via their contact with the catalyst in the liquid phase in the presence of one or more solvents at temperatures ranging from above 0 to 150° C. for a period of time ranging from instant contact to longer than 24 hours when the catalyst is to be stored in the treatment solution where the gas phase over the catalyst treatment solution during the treatment is air and no precautions were taken to remove the dissolve oxygen from the treatment solution. Both Raney-type and supported-type catalysts can be modified in this fashion.

The Ni, Cu, Co and mixtures thereof catalysts can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and organic molecules that interact strongly with metal surfaces via their contact with the catalyst in the liquid phase in the presence of one or more solvents at temperatures ranging from above 0 to 150° C., where the addition of one or more bases before, during and/or after the addition of the modifying agent is used. Both Raney-type and supported-type catalysts can be modified in this fashion.

The Ni, Cu, Co and mixtures thereof catalysts can be modified via the deposition of carbon containing residues with formaldehyde via its contact with the catalyst in the liquid phase in the presence of one or more solvents at temperatures ranging from above 0 to 150° C. for a period of time ranging from instant contact to longer than 24 hours when the catalyst is to be stored in the treatment solution where the gas phase over the catalyst treatment solution during the treatment is air and no precautions were taken to remove the dissolve oxygen from the treatment solution. Both Raney-type and supported-type catalysts can be modified in this fashion.

The Ni, Cu, Co and mixtures thereof catalysts can be modified via the deposition of carbon containing residues with formaldehyde via its contact with the catalyst in the liquid phase in the presence of one or more solvents at temperatures ranging from above 0 to 150° C. where the addition of one or more bases before, during and/or after the addition of formaldehyde is used. Both Raney-type and supported-type catalysts can be modified in this fashion.

The Ni, Cu, Co and mixtures thereof catalysts can be modified via the deposition of carbon containing residues with formaldehyde via its contact with the catalyst in the liquid phase in the presence of one or more solvents at temperatures ranging from above 0 to 150° C. where the addition of NaOH, KOH and their mixtures before, during and/or after the addition of formaldehyde is used. Both Raney-type and supported-type catalysts can be modified in this fashion.

The Ni, Cu, Co and mixtures thereof catalysts can be modified via the deposition of carbon containing residues with metal salt formates via their contact with the catalyst in the liquid phase in the presence of one or more solvents at temperatures ranging from above 0 to 150° C. Both Raney-type and supported-type catalysts can be modified in this fashion.

The Ni, Cu, Co and mixtures thereof catalysts can be modified via the deposition of carbon containing residues with metal salt formates via their contact with the catalyst in the liquid phase in the presence of one or more solvents at temperatures ranging from above 0 to 150° C. where the addition of one or more bases before, during and/or after the addition of sodium formate. Both Raney-type and supported-type catalysts can be modified in this fashion.

The Ni, Cu, Co and mixtures thereof catalysts can be modified via the deposition of carbon containing residues with metal salt formates via their contact with the catalyst in the liquid phase in the presence of one or more solvents at temperatures ranging from 70 to 130° C. Both Raney-type and supported-type catalysts can be modified in this fashion.

The Ni, Cu, Co and mixtures thereof catalysts can be modified via the deposition of carbon containing residues with metal salt formates via their contact with the catalyst in the liquid phase in the presence of one or more solvents at temperatures ranging from 70 to 130° C. where the addition of one or more bases before, during and/or after the addition of sodium formate. Both Raney-type and supported-type catalysts can be modified in this fashion.

The Cu catalysts can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. for a period of time ranging from instant contact to longer than 24 hours when the catalyst is to be stored in the treatment solution, whereby the gas phase over the catalyst treatment solution during the treatment can be air and/or inert gas. Both Raney-type and supported-type catalysts can be modified in this fashion.

The Cu catalysts can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. where the addition of one or more bases before, during and/or after the addition of the modifying agent is used. The gas phase over the catalyst treatment solution during the treatment can be air and/or inert gas Both Raney-type and supported-type catalysts can be modified in this fashion.

The Cu catalysts can be modified via the deposition of carbon containing residues with formaldehyde via its contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Cu catalysts can be modified via the deposition of carbon containing residues with formaldehyde via its contact with the catalyst in the liquid phase in the presence of one or more solvents at temperatures ranging from above 0 to 150° C. where the addition of one or more bases before, during and/or after the addition of formaldehyde is used. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. where the addition of one or more bases before, during and/or after the addition of the modifying agent is used. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with materials such as formaldehyde, metal salt formates, carbon monoxide, carbon dioxide, aldehydes, ketones, amides (such as formamide), carboxylic acids, salts of carboxylic acids and other organic molecules that interact strongly with metal surfaces via their contact with the catalyst in the liquid phase in the presence of one or more solvents at temperatures ranging from above 0 to 150° C. where the addition of one or more bases before, during and or after the addition of the modifying agent is used. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with formaldehyde via its contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with formaldehyde via its contact with the catalyst in the liquid phase in the presence of one or more solvents at temperatures ranging from 5 to 130° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with formaldehyde via its contact with the catalyst in the liquid phase in the presence of one or more solvents at temperatures ranging from above 0 to 150° C. where the addition of one or more bases before, during and/or after the addition of formaldehyde is used. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with formaldehyde via its contact with the catalyst in the liquid phase in the presence of one or more solvents at temperatures ranging from above 0 to 150° C. where the addition of one or more bases before, during and/or after the addition of formaldehyde is used. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with sodium formate via its contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with sodium formate via its contact with the catalyst in the liquid phase in the presence of one or more solvents at temperatures ranging from above 0 to 150° C. for a period of time ranging from instant contact to longer than 24 hours when the catalyst is to be stored in the treatment solution. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with sodium formate via its contact with the catalyst in the liquid phase in the presence of one or more solvents at temperatures ranging from above 0 to 150° C. where the addition of one of more bases before, during and/or after the addition of sodium formate. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with sodium formate via its contact with the catalyst in the liquid phase in the presence of one or more solvents at temperatures ranging from above 0 to 150° C. where the addition of one of more bases before, during and/or after the addition of sodium formate. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with sodium formate via its contact with the catalyst in the liquid phase in the presence of one or more solvents at temperatures ranging from 70 to 130° C. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with sodium formate via its contact with the catalyst in the liquid phase in the presence of one or more solvents at temperatures ranging from 70 to 130° C. Both Raney-type and supported-type catalysts can be modified in this fashion.

The Ni, Cu, Co and mixtures thereof catalysts promoted with one or more of the elements from the groups of Mo, Cr, Fe, Re and V can be modified via the deposition of carbon containing residues with sodium formate via its contact with the catalyst in the liquid phase in the presence of one or more solvents at temperatures ranging from 70 to 130° C. where the addition of one or more bases before, during and/or after the addition of sodium formate. Both Raney-type and supported-type catalysts can be modified in this fashion.

The Ni, Cu, Co and mixtures thereof catalysts promoted with one or more of the elements from the periodic groups of 1A, 2A, IIIB, IVB, VB, VIIB, VIIB, VIII, IB, IIB, IIIA, IVA, VA, VIA and the rare earth elements can be modified via the deposition of carbon containing residues with sodium formate via its contact with the catalyst in the liquid phase in the presence of one or more solvents at temperatures ranging from 70 to 130° C. where the addition of one or more bases before, during and/or after the addition of sodium formate. Both Raney-type and supported-type catalysts can be modified in this fashion.

The Ni, Cu, Co and mixtures thereof fixed bed catalysts can be modified in the liquid phase.

The Ni, Cu, Co and mixtures thereof fixed bed catalysts can be modified in the liquid phase, whereas the modification is carried out in a flooded fix bed reactor either with or without recirculation.

The Ni, Cu, Co and mixtures thereof fixed bed catalysts can be modified, whereas the modification is performed in the trickle phase in a fixed bed reactor and that the atmosphere surrounding the catalyst is either an inert atmosphere or a reducing atmosphere.

The Ni, Cu, Co and mixtures thereof fixed bed catalysts can be modified, whereas the modification is performed with an aerosol of the modifying agent in a fixed bed reactor and that the atmosphere surrounding the catalyst is either an inert atmosphere or a reducing atmosphere.

The Ni, Cu, Co and mixtures thereof fixed bed catalysts can be modified, whereas the modification is performed in the gas phase with the modifiers that are able to exist in the gas phase under the modifying conditions in a fixed bed reactor and that the atmosphere surrounding the catalyst is either an inert atmosphere or a reducing atmosphere.

The Ni, Cu, Co and mixtures thereof fixed bed catalysts can be modified, whereas the modification is performed in the gas phase with the modifiers of claim one that are able to exist in the gas phase under the modifying conditions in a fixed bed reactor where the treatment temperature can range from −50 to 500° C. and that the atmosphere surrounding the catalyst is either an inert atmosphere or a reducing atmosphere.

The Ni, Cu, Co and mixtures thereof fixed bed catalysts can be modified, whereas the modification temperature can range from above 0 to 150° C.

The Ni, Cu, Co and mixtures thereof catalysts, modified according the process of the invention can be are embedded in fatty amines.

The Ni, Cu, Co and mixtures thereof catalysts, modified according the process of the invention can be embedded in primary fatty amines.

The catalysts can be modified, whereas the catalytic metals are Fe, Pd, Pt, Ru, Ag, Au, V, Re, W, Mo, Rh, Ir and mixtures thereof.

The catalysts can be modified, whereas the modification is performed in the presence of a templating agent.

The catalysts can be modified, whereas the carbon depositing modification is performed under a reducing atmosphere.

The modified catalysts can be used for the improved stereoselective hydrogenation of compounds.

The modified catalysts can be used for the improved stereoselective hydrogenation of fructose to generate a higher concentration of mannitol.

The modified catalysts can be used for the improved chemoselective hydrogenation of compounds.

The modified catalysts can be used for the improved chemoselective hydrogenation of compounds where the more strongly adsorbed moeity is hydrogenated over the less strongly adsorbed one.

The modified catalysts can be used for the improved chemoselective hydrogenation of compounds where nitrites are preferentially hydrogenated over olefins.

The modified catalysts can be used for the improved chemoselective hydrogenation of compounds where carbonyls are preferentially hydrogenated over olefins.

The modified catalysts can be used for the improved chemoselective hydrogenation of compounds where unsaturated fatty amines are produced from unsaturated fatty nitrites.

The modified catalysts can be used for the improved regioselective hydrogenation of compounds.

The modified catalysts can be used for the improved regioselective hydrogenation of compounds where the less sterically hindered moeity is preferentially hydrogenated over the more sterically hindered one.

The modified catalysts can be used for the improved regioselective hydrogenation of compounds where exocyclic moieities are preferentially hydrogenated over endocyclic ones.

The modified catalysts can be used for the improved regioselective hydrogenation of compounds where terminal moeities are preferentially hydrogenated over internal ones.

The modified catalysts can be used for the improved regioselective hydrogenation of compounds where moeities with fewer and less bulkier groups attached to them are preferentially hydrogenated over moeities with more and/or bulkier groups attached to them.

The modified catalysts can be used for the improved hydrogenation of nitrites to primary amines.

The modified catalysts can be used for the improved conversion of nitrites to the corresponding dimerized disubstituted imines.

The modified catalysts can be used for the improved hydrogenation of nitrites to primary amines in the presence of ammonia.

The modified catalysts can be used for the improved hydrogenation of nitriles to primary amines in the presence of one or more bases.

The modified catalysts can be used for the improved hydrogenation of nitrites to primary amines in the presence of NaOH, KOH or their mixtures.

The modified catalysts can be used for the improved hydrogenation of aromatic nitrites to primary amines.

The modified catalysts can be used for the improved hydrogenation of aromatic nitrites to primary amines in the presence of ammonia.

The modified catalysts can be used for the improved hydrogenation of aromatic nitrites to primary amines in the presence of bases.

The modified catalysts can be used for the improved hydrogenation of alpha, omega dinitriles to amino nitrites.

The modified catalysts can be used for the improved hydrogenation of nitro groups without the formation of dimers.

The modified catalysts can be used for the improved hydrogenation of compounds where the performance of the catalyst is fitted to the capabilities of the reactor.

The modified catalysts can be used towards the hydrogenation of triglycerides, where their olefin moeities, as monitored by the molecule's iodine value, are hydrogenated to provide either totally saturated triglycerides or triglycerides of a certain level of unsaturation as determined upon the hydrogenation process, the design of the catalyst and the reaction conditions such as temperature and hydrogen pressure.

The reactions with a modified catalyst according to the invention can be performed in either the liquid phase, the gas phase, the trickle phase or in an aerosol of the reactant(s) via either a single pass or a recycling process in either a stirred tank reactor, a fluidized bed reactor or a fixed bed reactor as determined by the best fit to the reaction type and catalyst technology.

The method of this invention is also found to be useful in the hydrogenation of nitrites to primary amines without the formation of secondary or tertiary amines. These nitrites may either be aromatic or alphatic in nature. A special case would be the selective hydrogenation of dinitriles to diamines or aminonitriles without the formation of ring-shaped secondary amines, linear secondary amines or tertiary amines in general.

As with other catalytic nitrile hydrogenations, hydrogenating the nitrile with one of the catalysts of this invention in the presence of ammonia helps the selectivity of the reaction. However, the amount of ammonia needed for the desired primary amine selectivity is lower and the level of primary amine selectivity enhancement is higher with the catalysts of this invention than with the standard catalysts used in the industry.

In general, the invented catalysts are preferred for organic transformations where the formation of dimers and oligiomeres are not desired.

Thus the invented catalyst can be used in many reactions, such as the hydrogenation of nitro groups, where the formation of dimers and other undesired bulky compounds can be avoided.

The catalysts of this invention can also be used for the avoidance of bulky intermediates on the catalysts' surface resulting in a higher than usual yield of the desired compound.

It is known in the industry that a pyrophoric and/or an air sensitive catalyst (i.e., a Raney-type catalysts and other types of reduced metal catalysts) can be protected from air via embedding them in either a waxy, fatty, polyglycol type or other embedding type of materials. One common type of embedding agent would be a secondary fatty amine such as distearylamine whose correspondingly embedded catalyst is commonly used for the hydrogenation of fatty nitrites to primary fatty amines.

In some cases, the presence of the secondary fatty amine embedding agent may cause the primary amine product to be slightly turbid thereby limiting its use in some applications. This could be remedied by the use of a primary fatty amine as an embedding agent, however primary fatty amines tend to form secondary fatty amines and ammonia during the embedding process. Not only does this create the above mentioned turbidity problem, but it also creates health and safety issues as the resulting embedded catalyst gives off large amounts of ammonia during not only the embedding process, but also during storage.

Thus, any catalyst that could be embedded in primary amines without the formation of secondary amines and ammonia would be advantageous for the use of fatty nitrile hydrogenation and for any other hydrogenation where the primary fatty amine is preferred over the secondary fatty amine as an embedding agent.

The modified catalyst (treated with formaldehyde or any of the other previously mentioned modifying agents) of this patent is such a catalyst where embedding it in the primary fatty amine generates far less secondary fatty amine than the unmodified catalyst.

Hence one part of this invention is the use of the modified catalyst of this patent for the production of a primary fatty amine embedded catalyst. The modification with the previously mentioned modifying agents can be performed prior and/or during the embedding process.

The invention of this patent could be used to modify the activity of a catalyst, so that it fits within the mass transfer limitations of an existing reactor. In this way, the formation of undesirable coke on the metal surface via hydrogen deficiency can be inhibited.

In another respect, the controlled deposition of carboneaceous materials on the catalytic surface via the invention of this patent can also be used to control and inhibit the formation of coke on the catalytic surface and the progress of catalyst deactivation via the templating of the free active surface and the avoidance of catalytic centers that may produce irreversible coke precursors and other catalyst poisons.

Activated metal catalysts are also known in the fields of chemistry and chemical engineering as Raney-type, sponge and/or skeletal catalysts. They are used, largely in powder form, for a large number of hydrogenation, dehydrogenation, isomerization and hydration reactions of organic compounds. These powdered catalysts are prepared from an alloy of a catalytically-active metal, also referred to herein as the catalyst metal, with a further alloying component which is soluble in alkalis.

Mainly nickel, cobalt, copper, or iron are used as catalyst metals.

Aluminum is generally used as the alloying component which is soluble in alkalis, but other components may also be used, in particular zinc and silicon or mixtures of these either with or without aluminum.

These so-called Raney alloys are generally prepared by the ingot casting process. In that process a mixture of the catalyst metal and, for example, aluminum are first melted and casted into ingots. Typical alloy batches on a production scale amount to about ten to one hundred kg per ingot. According to DE 21 59 736 cooling times of up to two hours were obtained. This corresponds to an average rate of cooling of about 0.2 K/s. In contrast to this, rates of 102 to 106 K/s and higher are achieved in processes where rapid cooling is applied (for example an atomizing process).

The rate of cooling is affected in particular by the particle size and the cooling medium (see Materials Science and Technology edited by R. W. Chan, P. Haasen, E. J. Kramer, Vol. 15, Processing of Metals and Alloys, 1991, VCH-Verlag Weinheim, pages 57 to 110). A process of this type is used in EP 0 437 788 B 1 in order to prepare a Raney alloy powder. In that process the molten alloy at a temperature of 5 to 500° C. above its melting point is atomized and cooled using water and/or a gas.

The invention of this patent can be applied to the catalysts prepared from slowly, moderately and rapidly cooled alloys. The use of cooling mediums, including but not limited to water, air and inert gases (e.g., Ar, He, N₂ and others) can also be used in fabricating the alloys that are activated with caustic solutions in order to generate the catalysts being modified with the above mentioned modifiers.

To prepare a catalyst, the Raney alloy is first finely milled, if it has not been produced in the desired powder form during preparation. Then the aluminum is partly (and if need be, totally) removed by extraction with alkalis such as, for example, caustic soda solution (other bases such as KOH are also suitable) to activate the alloy powder. Following extraction of the aluminum, the remaining catalytic power has a high specific surface area (BET), between 5 and 150 m2/g, and is rich in active hydrogen.

The activated catalyst powder is pyrophoric and stored under water or organic solvents or is embedded in organic compounds which are solid at room temperature.

These catalysts can also be promoted with one or more elements coming from the periodic groups 1A, 2A, IIIB, IVB, VB, VIIB, VIIB, VIII, IB, IIB, IIIA, IVA, VA, VIA and the rare earth elements. Preferably the promoting elements come from the periodic groups IIIB, IVB, VB, VIIB, VIIB, VIII, IB, IIB, IIIA, IVA and VA.

One or more of these promoting elements can be incorporated into the catalyst by either initially adding the element(s) to the precursor alloy before leaching or by adsorbing the element(s) either during or after the activation of the catalyst.

Another option is to incorporate one or more promoter elements into the alloy and to adsorb additionally one or more of either the same and/or different promoter elements either during or after the activation of the alloy with caustic or any other suitable base. These promoted catalysts were found to respond exceptionally well to the treatment procedures (e.g., treatment with formaldehyde, sodium formate and the other mentioned modifiers) of this patent in order to produce very selective catalysts.

This modification procedure can also be applied to the various forms of activated base metal fixed bed catalysts found in the literature. Examples of these fixed bed forms include, but are not limited to, tablets (Schütz et al. EP648535, Freund et al. DE19721898, Ostgard et al. U.S. Pat. No. 6,489,521, Ostgard et al. U.S. Pat. No. 6,284,703), extrudates (Sauer et al. EP 0880996 and Cheng et al. U.S. Pat. No. 4,826,799), activated hollow spheres (Ostgard et al. DE10101647, Ostgard et al. DE10101646, Ostgard et al. DE10065031, Ostgard et al. U.S. Pat. No. 6,486,366, Ostgard et al. U.S. Pat. No. 6,437,186, Ostgard et al. EP1068900), activated flake or fiber forms (e.g., tablets and mats in Ostgard et al. EP106B896), granules (formed by the agglomeration of alloy powders with binders and pore builders), supported activated catalytic metal/Al alloys, activated Al treated catalytic metal sheets and activated Al treated monoliths containing a catalytic metal that can alloy with the Al and can be activated with caustic to the catalyst.

Raney-type fixed bed catalysts can also be made by the leaching (e.g., via caustic activation or the use of any other suitable bases and their combinations) of chunks of alloy consisting of base metals with optionally promoters and alkali leachable metals such as Al, Zn, Si or combinations thereof.

The precursor alloy chunks can be formed by the coarse grinding of casted slowly cooled alloys, the controlled solidification of gas (e.g., nitrogen or air) cooled alloys, the controlled solidification of liquid (e.g., water) cooled alloys or the controlled solidification of gas and liquid cooled alloys. Such controlled cooling processes include the cooling of the alloy melt to about 5 to 200° C., or preferably 10 to 100° C., above the solidification temperature before introducing it into the liquid or gas cooling medium. The chunks can then be formed by either adding the cooled melt to the cooling medium (e.g., water) dropwise where the drop and corresponding chunk size depends on the size of the opening used for the formation of the drop or in a continuous stream that may be interrupted mechanically to the right chunk size before the alloy is quenched. The final, initial or combined cooling rates of these chunks of alloy may vary from 0.1 to 106 via the methods mentioned above.

The above mentioned chunks of alloy may be activated by caustically leaching away the desired amount of Al followed by treatment with a compound (e.g., formaldehye, sodium formate, carbon monoxide and others) that will lead to the controlled deposition of a carbonaceous residue on the catalyst's surface in order to produce the catalyst of the present invention. Optionally the catalyst may be washed between the activation and deposition steps.

As described by for the powder catalyst, all of the above mentioned fixed bed catalyst can also be promoted with one or more elements coming from the periodic groups 1A, 2A, IIIB, IVB, VB, VIIB, VIIB, VIII, IB, IIB, IIIA, IVA, VA, VIA and rare earth elements.

Preferably the promoting elements come from the periodic groups IIIB, IVB, VB, VIIB, VIIB, VIII, IB, IIB, IIIA, IVA and VA.

One or more of these promoting elements can be incorporated into the catalyst by either initially adding the element(s) to the precursor alloy before leaching or by adsorbing the element(s) either during or after the activation of the catalyst.

Another option is to incorporate one or more promoter elements into the alloy and to adsorb additionally one or more of either the same and/or different promoter elements either during or after the activation of the alloy with caustic or any other suitable base.

These promoted catalysts were found to respond exceptionally well to the treatment procedures (e.g., treatment with formaldehyde, sodium formate and the other mentioned modifiers) of this patent in order to produce very selective catalysts.

As in the case of the powder catalysts, the fixed bed catalysts can be treated with a carbon depositing material (e.g., formaldehye, sodium formate, carbon monoxide and others) in the slurry phase, in the trickle phase and/or in the gas phase over a broad range of conditions.

These modifications with the above mentioned modifiers (e.g., formaldehye, sodium formate, carbon monoxide and others) can be also carried out in a fixed bed reactor before or during the desired reaction.

This modification can also be carried out during the reaction in the case of the slurry phase powder catalysts as well.

The fixed bed catalysts can also be modified with the above mentioned modifiers (e.g., formaldehye, sodium formate, carbon monoxide and others) in a specially outfitted autoclave, where the fixed bed catalyst is thoroughly mixed with the treatment medium. Such methods may involve outfitting the stirred tank reactor with a basket to hold the catalyst, that has the modifying solution forced through it via the stirrer, or the basket with the catalyst may be a part of the stirrer itself where the catalyst basket is rotated through the modification solution.

This catalyst is then modified with the above mentioned modifiers in either the gas, trickle or slurry phase, or with any type of aerosol of the modification solution in order to generate the modified catalyst of this invention. The modification temperature can range from below room temperature to temperatures much higher as determined by the properties of the catalyst, the solvent and/or the modifier.

When the catalyst is modified in the gas phase, its modification temperature can reach much higher temperatures such as 500 to 1000° C.

If the modifying agent is not a gas, its use for this purpose can be applied as an aerosol and, if needed, in an appropriate solvent. This modification can also take place in the trickle phase.

In the gas phase, catalyst modification can also occur at lower temperatures (even at subambient e.g. −100% with CO and −50% for CO₂) as determined by the conditions and the condensation properties of the modifier. The preferred modification conditions range from 5 to 130° C. in an aqueous solution of the modifier where the concentration of the modifier is dependent on the amount of desired modifier per gram of catalyst.

It is also possible to control this modification procedure further by performing the modification at subambient temperatures (i.e., below room temperature) above the freezing point of the medium. In such situations, the temperature can be ramped in a controlled way to the desired modification treatment so that the modifiers can be homogeneous distributed before modification

One type of modification method involves the introduction of the modifier to the catalyst at a temperature below the decomposition temperature, the homogenization of the catalyst modifier mixture and the ramping of the system temperature to that above the modifier decomposition temperature. In this way the modification can occur in a very controlled manner. Similar controlled methods can be used involving the temperature in which a modifier dissolves in the used solvent system, where the initial contact of the modifier with the catalyst is controlled by its dissolution into the solvent.

An example of this would be to start the aqueous formaldehyde modification of the catalyst at temperatures above 0° C. (e.g., about 5° C.).

When other solvent systems (e.g., organic solvents, mixed solvents, solvents with other solutes and other systems) or gas systems are used (e.g., CO) even lower temperatures could be used for this modification.

The modification according to the invention can be performed by adding the catalyst to the medium containing the modifying agent, adding the modifying agent to a medium containing the catalyst, adding additional modifier to a modified catalyst in a medium, adding additional catalyst to a modified catalyst in a medium or any reasonable variant of the aforementioned modification methods.

Our results have also shown that the concentration of the modifier in the modification solution is not a critical issue, and the catalyst may be modified with either a relatively concentrated or dilute medium containing the compound to be decomposed on the active metal surface.

In the case of formaldehyde, the modification process starts as soon as the modifier comes in contact with the catalyst and modification times less than 15 minutes are also effective.

It is also possible to modify all the Raney-type catalysts mentioned above during their basic activation as the Al is being leached out with caustic or any other suitable bases (e.g., KOH) and their mixtures.

In addition to the above mentioned catalysts, according to the present invention, both powder and fixed bed supported Ni, Co, Cu, Fe, Pd, Pt and Ru catalysts can be modified by the above mentioned modifiers (e.g., formaldehye, sodium formate, carbon monoxide and others) in order to change their selectivities in the above mentioned reactions.

The above and other objects of the invention are achieved by producing a modified catalyst via the treatment of an activated base metal catalyst (i.e., a Raney-type base metal catalyst) or a supported metal catalyst with a modifying agent that deposits carbon containing residues on a catalytic surface. These carbon containing residues may also contain oxygen, sulfur, nitrogen, hydrogen and other atoms that are commonly present in organic molecules.

The preferred precursors for the deposition of carbon residues include formaldehyde and metal salt formates. However, other molecules such as carbon monoxide, carbon dioxide, aldehydes, ketones, amides (such as formamide), other carboxylic acids, salts of other carboxylic acids and other organic molecules that interact strongly with activated base metal surfaces can be used.

The treatment temperature is preferably between the temperatures of 0 and 150° C. and it can be performed in the slurry, trickle and/or gas phase, as well as with any type of aerosols of the modifier.

Common organic solvents may also be used in the modification procedure.

In the slurry phase the modification can also be performed in an aqueous suspension of the catalyst. The slurry phase modification of the catalyst can also be performed with organic solvents as well.

Suitable organic solvents in which, in addition to water, the modification may be carried out include aliphatic hydrocarbons (e.g. pentane and hexane), aromatic hydrocarbons (e.g. benzene and toluene), alkanols (e.g. methanol, ethanol, and propanol), aliphatic and cyclic ethers (e.g. diethyl ether and, respectively, tetrahydrofuran, and dioxan), as well as heteroaromatics (e.g. pyridine). The dispersion medium can consists of water alone or of a single organic solvent or of two or more of such liquids. For example, an aqueous alkanol, e.g. aqueous ethanol, can be used as the liquid dispersion medium. In general, the process is carried out by dispersing the hydrogenation catalyst in water and/or such an organic solvent, because a dissolution of the catalyst does not take place due to its nature. On the other hand, the modification agent must at least partially dissolve in the dispersion medium.

One variant of the current modification methods of this invention avoids the presence of dissolved nickel by the timely addition of one or more bases (e.g., NaOH, KOH, organic bases and others) to the modification medium thereby decreasing the cost of waste disposal. This modification can be carried out in the presence or the absence of inert gases, where avoiding the use of a inert gas will make this process more cost effective.

This modifier is applied to the catalyst so as to improve its stereoselectivity, chemoselectivity, regioselectivity in organic transformations as well as in reactions where the avoidance of dimerization and oligiomerization for the desired reaction results are necessary.

The catalyst of this invention is preferred for hydrogenations where one of the above mentioned selectivities are required. Such hydrogenations include, but are not limited to, the stereoselective hydrogenation of ketoses, the chemo- and regioselective formation of unsaturated fatty amines from unsaturated fatty nitriles, the selective hydrogenation of nitriles to primary amines, the selective hydrogenation of dinitriles to diamines or aminonitriles, the chemoselective hydrogenation of unsaturated ketones to unsaturated alcohols, the chemo- and regioselective hydrogenation of unsaturated aldehydes to unsaturated alcohols, and the regioselective hydrogenation of terminal olefins over the more heavily substituted ones.

Other reactions (e.g., the hydrogenation of aromatic and aliphatic nitro groups) that can form dimers oligomers or bulk intermediates and side products are preferably performed with the catalyst of this invention so as to avoid these undesired compounds.

The precursor catalyst, prior to modification via carbon residue deposition, can be leached from rapidly cooled alloys with and without special mediums, as well as, normally casted and slowly cooled alloys of all types. The precursor catalyst optionally contains promoting elements from the periodic groups 1A, 2A, IIIB, IVB, VB, VIIB, VIIB, VIII, IB, IIB, IIIA, IVA, VA, VIA and the rare earth elements. Preferably the promoting elements come from the periodic groups IIIB, IVB, VB, VIIB, VIIB, VIII, IB, IIB, IIIA, IVA, VA and the rare earth elements.

One or more of these promoting elements can be incorporated into the catalyst by either initially adding the element(s) to the precursor alloy before leaching or by adsorbing the element(s) either during or after the activation of the catalyst.

Promotion with combinations of the above mentioned elements can also be accomplished by using a combination of techniques where one or more element(s) are added into the alloy and the other(s) or more of the same are/is added during or after leaching the alloy with caustic solutions.

Promotion with one or more of the above mentioned elements can take place either during or after modification.

The catalyst of the invention can be either a fixed bed or power catalysts. The common fixed bed forms include extrudates, tablets, granules, activated chunks where the original alloy was solidified in a controlled way (slowly, rapidly and/or combinations thereof), hollow spheres, hollow extrudates, fiber/flake tablets/mats, monoliths, metal sheets and supported Raney-type catalysts.

The catalysts can be treated and used in the slurry phase, trickle phase, gas phase, in various types of aerosols of the modifier and/or combinations thereof.

Modification of the catalyst can also be carried out during the reaction. If deposited material from the modification is removed during the reaction, this modification treatment could then be repeated to generate the desired properties of the invented catalyst. This rejuvenation process can be carried out in the reactor used by the reaction or it can be carried out in a separate reactor.

This modification method can also be carried out in the presence of other adsorbed molecules, so that better control of the carbonaceous structure can be achieved. In this way a templating effect could be realized on the surface of the catalyst. The templating agent can be for example, but not limited to, Sorbitol, Glucose, Mannitol, Mannose, tartaric acid.

Another part of this invention is the embedding of the above mentioned modified catalysts in primary fatty amines where the conversion of the primary fatty amines to secondary fatty amines and ammonia during the embedding process and storage is clearly lower than with unmodified catalysts. These embedded catalysts are clearly preferred for the hydrogenation of fatty nitriles to primary fatty amines where turbidity issues are important.

In addition to Raney-type catalysts, this modification method may also be applied to supported powder and fixed bed base and precious metal supported catalysts as well as combinations thereof.

### EXAMPLE 1

### Treatment of Raney-Type Ni Catalysts with Formaldehyde

A commercially available Raney-type nickel catalyst with an average particle size of ˜53 μm is treated with different amounts of formaldehyde according to the procedure described here.

The desired quantities of the catalyst are suspended in an aqueous solution containing formaldehyde. This suspension is stirred for 1 hour at room temperature under air or nitrogen.

After treatment, the catalyst is allowed to settle, the overstanding aqueous solution is decanted, and the catalyst is washed three times with distilled water. The specific conditions used for the preparation of each catalyst are given in detail in Table 1.

TABLE 1
Conditions for the formaldehyde treatment of Raney-type Ni catalysts.

| | | Grams | | | | | | water for |
|---|---|---|---|---|---|---|---|---|
| | mmol | Catalyst | | aqueous | gas | | | each |
| Catalyst | formaldehyde | (dry | Formaldehyde | solution | above | Temperature | Time | washing |
| N^{o} | per g cat | basis) | (g) | (ml) | solution | (° C.) | (h) | step (ml) |
| E1a | 0.27 | 32.35 | 0.259 | 141 | air | RT | 1 | 100 |
| E1b | 0.50 | 32.35 | 0.485 | 141 | air | RT | 1 | 400 |
| E1c | 0.76 | 110 | 2.516 | 238 | air | RT | 1 | 880 |
| E1d | 0.76 | 110 | 2.516 | 238 | nitrogen | RT | 1 | 880 |
| E1e | 1.00 | 32.35 | 0.971 | 141 | air | RT | 1 | 400 |
| E1f | 1.29 | 65 | 2.516 | 283 | air | RT | 1 | 880 |
| E1g | 1.29 | 65 | 2.516 | 283 | nitrogen | RT | 1 | 880 |
| E1h | 1.66 | 65 | 3.23 | 263 | air | RT | 1 | 880 |
| E1i | 1.66 | 65 | 3.23 | 141 | air | RT | 1 | 880 |
| E1j | 2.16 | 130 | 8.41 | 283 | air | RT | 1 | 1600 |
| E1k | 2.65 | 130 | 10.35 | 283 | air | RT | 1 | 1600 |

RT = room temperature

### EXAMPLE 2

### Formaldehyde Treatment of Raney-Type Ni Catalysts where the Precursor Alloys Contains Mo

27.7 g (on a dry basis) of a catalyst, whose precursor alloy contained Mo, are suspended in 121 ml of an aqueous solution containing formaldehyde. This suspension is stirred for 1 hour at room temperature under air. After treatment, the catalyst is allowed to settle, the overstanding aqueous solution is decanted and the catalyst is washed three times with 400 ml of distilled water. The specific conditions used for the preparation of each catalyst are given in detail in Table 2.

TABLE 2
Conditions for the formaldehyde treatment of Raney-type Ni catalysts whose precursor
alloys contained Mo.

| | mmol | Grams | | aqueous | gas | | | water for |
|---|---|---|---|---|---|---|---|---|
| Catalyst | formaldehyde | Catalyst | Formaldehyde | solution | above | Temperature | | each washing |
| N^{o} | per g cat | (dry basis) | (g) | (ml) | solution | (° C.) | Time (h) | step (ml) |
| E2a | 0.33 | 27.7 | 0.277 | 121 | air | RT | 1 | 400 |
| E2b | 0.67 | 27.7 | 0.554 | 121 | air | RT | 1 | 400 |
| E2c | 0.77 | 27.7 | 0.6371 | 121 | air | RT | 1 | 400 |
| E2d | 1.30 | 27.7 | 1.080 | 121 | air | RT | 1 | 400 |
| E2e | 1.67 | 27.7 | 1.385 | 121 | air | RT | 1 | 400 |
| E2f | 2.00 | 27.7 | 1.662 | 121 | air | RT | 1 | 400 |
| E2g | 2.33 | 27.7 | 1.939 | 121 | air | RT | 1 | 400 |
| E2h | 2.67 | 27.7 | 2.216 | 121 | air | RT | 1 | 400 |
| E2i | 3.00 | 27.7 | 2.493 | 121 | air | RT | 1 | 400 |
| E2j | 3.34 | 27.7 | 2.773 | 121 | air | RT | 1 | 400 |
| E2k | 4.00 | 27.7 | 3.323 | 121 | air | RT | 1 | 400 |

RT = room temperature.

### EXAMPLE 3

### Treatment of a Raney-Type Ni Catalyst with Sodium Formate

32.35 g (on a dry basis) of a Raney-type Ni catalyst with an average particle size of ˜53 μm are suspended in 141 ml of an aqueous solution containing sodium formate. This suspension is stirred for 1 hour at either 25 or 90° C. under air. After modification, the treated suspension is allowed to cool (if needed) as the catalyst settled to the bottom of the vessel, the overstanding aqueous solution is decanted and the catalyst is washed three times with distilled water. The specific conditions used for the preparation of these catalysts are given in detail in Table 3.

TABLE 3
Conditions for the sodium formate treatment of a Raney-type Ni catalyst.

| | mmol Na | | | | | | | |
|---|---|---|---|---|---|---|---|---|
| | formate | Grams | | | | | | water for |
| | per g | Catalyst | sodium | aqueous | gas | | | each |
| Catalyst | catalyst on | (dry | formate | solution | above | Temperature | | washing |
| N^{o} | dry basis | basis) | (g) | (ml) | solution | (° C.) | Time (h) | step (ml) |
| E3a | 1.66 | 32.35 | 3.65 | 141 | air | 90 | 1 | 400 |
| E3b | 4.00 | 32.35 | 8.8 | 141 | air | 90 | 1 | 400 |
| E3c | 12.0 | 32.35 | 26.4 | 141 | air | 90 | 1 | 400 |
| E3d | 4.00 | 32.35 | 8.8 | 141 | air | 25 | 1 | 400 |
| E3e | 1.30 | 32.35 | 2.86 | 141 | air | 25 | 1 | 400 |

### EXAMPLE 4

### Treatment of Raney-Type Ni Catalysts, Whose Precursor Alloys Contains Mo, with Sodium Formate

27.7 g (on a dry basis) of a catalyst, whose precursor alloy contains Mo, are suspended in a 121 ml aqueous solution containing different amounts of sodium formate. This suspension is stirred for 1-5 hours at room temperature, at 90° or at 105° C., under air. After modification, the treated suspension is allowed to cool as the catalyst settled to the bottom of the vessel, the overstanding aqueous solution is decanted and the catalyst is washed three times with 400 ml of distilled water. The specific conditions used for the preparation of each catalyst are given in detail in Table 4.

TABLE 4
The sodium formate treatment conditions of Raney-type Ni catalysts, whose precursor
alloys contains Mo.

| | mmol Na | | | | | | | |
|---|---|---|---|---|---|---|---|---|
| | formate | | | | | | | |
| | per g | Grams | | | | | | water for |
| | catalyst | Catalyst | sodium | aqueous | gas | | | each |
| Catalyst | on dry | (dry | formate | solution | above | Temperature | | washing |
| N^{o} | basis | basis) | (g) | (ml) | solution | (° C.) | Time (h) | step (ml) |
| E4a | 1.30 | 27.7 | 2.45 | 121 | air | 90 | 1 | 400 |
| E4b | 1.67 | 27.7 | 3.14 | 121 | air | 90 | 1 | 400 |
| E4c | 2.00 | 27.7 | 3.77 | 121 | air | 90 | 1 | 400 |
| E4d | 2.33 | 27.7 | 4.39 | 121 | air | 90 | 1 | 400 |
| E4e | 2.67 | 27.7 | 5.02 | 121 | air | 90 | 1 | 400 |
| E4f | 3.00 | 27.7 | 5.65 | 121 | air | 90 | 1 | 400 |
| E4g | 3.29 | 27.7 | 6.20 | 121 | air | 90 | 1 | 400 |
| E4h | 2.00 | 27.7 | 3.77 | 121 | air | 90 | 2 | 400 |
| E4i | 2.00 | 27.7 | 3.77 | 121 | air | 90 | 3 | 400 |
| E4j | 2.00 | 27.7 | 3.77 | 121 | air | 90 | 5 | 400 |
| E4k | 2.00 | 27.7 | 3.77 | 121 | air | 105 | 1 | 400 |
| E4l | 1.67 | 27.7 | 3.14 | 121 | air | 90 | 5 | 400 |
| E4m | 3.00 | 27.7 | 5.65 | 121 | air | 90 | 5 | 400 |
| E4n | 3.29 | 27.7 | 6.20 | 121 | air | 90 | 5 | 400 |

### EXAMPLE 5

### Treatment of a Raney-Type Ni Catalyst with Formaldehyde

A Raney-type nickel catalyst with an average particle size of ˜28 μm is treated with formaldehyde according to the procedure described here. The desired quantity of the catalyst is suspended in an aqueous solution containing formaldehyde. This suspension is stirred for 1 hour at room temperature under air. After treatment, the catalyst is allowed to settle to the bottom of the vessel, the aqueous solution is decanted, and the catalyst is washed three times with distilled water. The specific conditions used for the preparation of this catalyst are given in detail in Table 5.

TABLE 5
Conditions for the formaldehyde treatment of a Raney-type Ni catalyst.

| | mmol | Grams | | aqueous | gas | | | water for |
|---|---|---|---|---|---|---|---|---|
| Catalyst | formaldehyde | Catalyst | Formaldehyde | solution | above | Temperature | | each washing |
| N^{o} | per g cat | (dry basis) | (g) | (ml) | solution | (° C.) | Time (h) | step (ml) |
| E5a | 1.30 | 118 | 4.603 | 515 | air | RT | 1 | 1600 |

RT = room temperature

### EXAMPLE 6

### Treatment of a Raney-Type Ni Catalyst with Sodium Formate

A Raney-type Ni catalysts with an average particle size of ˜28 μm is suspended in 141 ml of an aqueous solution containing sodium formate. This suspension is stirred for 1 hour at room temperature or 90° C. under air. After treatment, the modified suspension is allowed to cool (if originally heated) as the catalyst settled to the bottom of the vessel, the overstanding aqueous solution is decanted and the catalyst is washed three times with distilled water. The specific conditions used for the preparation of these catalysts are given in detail in Table 6.

TABLE 6
Conditions for the sodium formate treatment of a Raney-type Ni catalyst.

| | mmol Na | | | | | | | |
|---|---|---|---|---|---|---|---|---|
| | formate | | | | | | | |
| | per g | Grams | | | | | | water for |
| | catalyst | Catalyst | sodium | aqueous | gas | | | each |
| Catalyst | on dry | (dry | formate | solution | above | Temperature | | washing |
| N^{o} | basis | basis) | (g) | (ml) | solution | (° C.) | Time (h) | step (ml) |
| E6a | 1.30 | 118 | 10.43 | 514 | air | RT | 1 | 1600 |
| E6b | 1.30 | 29.5 | 2.607 | 129 | air | 90 | 1 | 400 |

RT = room temperature

### EXAMPLE 7

### Sodium Formate Treatment of a Raney-Type Ni Catalyst that is Post-Activation Doped with Mo

A Raney-type Ni catalyst, that is post-activation doped with Mo (via an ammonium molybdate compound) and has an average particle size of ˜28 μm, is suspended in an aqueous solution containing sodium formate. This suspension is stirred for 1 hour at room temperature under air. After treatment the catalyst in the modified suspension is allowed to settle to the bottom of the vessel, the overstanding aqueous solution is decanted and the catalyst is washed three times with distilled water. The specific conditions used for the preparation of this catalyst are given in detail in Table 7.

TABLE 7
Conditions for the sodium formate treatment of a Raney-type Ni catalyst that is
post-activation doped with Mo.

| | mmol Na | | | | | | | |
|---|---|---|---|---|---|---|---|---|
| | formate | | | | | | | |
| | per g | Grams | | | | | | water for |
| | catalyst | Catalyst | sodium | aqueous | gas | | | each |
| Catalyst | on dry | (dry | formate | solution | above | Temperature | | washing |
| N^{o} | basis | basis) | (g) | (ml) | solution | (° C.) | Time (h) | step (ml) |
| E7a | 1.30 | 87.3 | 7.71 | 380 | air | RT | 1 | 1400 |

RT = room temperature

### EXAMPLE 8

### Formaldehyde Treatment of a Raney-Type Ni Catalyst that Avoids the Presence of Dissolved Ni in the Treatment Solution

A Raney-type nickel catalyst with an average particle size of ˜53 μm is treated with formaldehyde according to the procedure described here so as to avoid the presence of dissolved Ni in the treatment suspension. The pH of an aqueous suspension containing ˜3200 grams of water and 1000 grams (on a dry basis) of the catalyst is adjusted to approximately 13 by the addition of 450 ml of a 5 wt % aqueous NaOH solution. A 500 ml solution containing 84.69 grams of formaldehyde is then added to the catalyst suspension over 30 minutes at room temperature. The suspension of the treated catalyst is allowed to stir for an additional hour at room temperature. At the end of this hour, a metal analysis of the catalyst treatment suspension shows, that the concentration of dissolved Ni is 0 ppm, and further examination indicates, that no formaldehyde remained in the treatment solution. The catalyst is then allowed to settle to the bottom of the vessel, the overstanding solution is decanted, and the catalyst is washed twice with 2000 ml of water. Since the treatment solution does not contain dissolved Ni or formaldehyde, the washing step is optional. All steps are performed under air and the conditions used for this catalyst preparation are given in Table 8.

TABLE 8
Conditions for the formaldehyde treatment of a Raney-type Ni catalyst that avoids
the presence of dissolved Ni in the treatment solution.

| | | ml of a 5 wt- | | | | |
|---|---|---|---|---|---|---|
| | | % NaOH | | | | |
| | | aqueous | | | | |
| | | solution | | | | |
| | Grams | added to the | | mmol | | |
| | Catalyst (dry | catalyst | Formaldehyde | formaldehyde | gas above | Temperature |
| Catalyst N^{o} | basis) | suspension | (g) | per g cat | solution | (° C.) |
| E8a | 1000 | 450 | 84.69 | 2.82 | air | RT |

RT = room temperature

### EXAMPLE 9

### Formaldehyde Treatment of a Raney-Type Ni Catalyst that Avoids the Presence of Dissolved Ni in the Treatment Solution

A Raney-type nickel catalyst with an average particle size of ˜53 μm is treated with formaldehyde according to the procedure described here so as to avoid the presence of dissolved Ni in the treatment suspension.

The pH of an aqueous suspension containing ˜640 grams of water and 200 grams (on a dry basis) of the catalyst was adjusted to approximately 13 by the addition of 48 ml of a 5 wt % aqueous NaOH solution. A 100 ml solution containing 10.16 grams of formaldehyde is then added to the catalyst suspension over 30 minutes at room temperature. The suspension of the treated catalyst is allowed to stir for an additional hour at room temperature. At the end of this hour, a metal analysis of the catalyst treatment suspension shows that the concentration of dissolved Ni is 0 ppm, and further examination indicates, that no formaldehyde remains in the treatment solution. The catalyst is then allowed to settle to the bottom of the vessel. The overstanding solution is decanted, and the catalyst is washed twice with 400 ml of water. Since the treatment solution does not contain dissolved Ni or formaldehyde, the washing step was optional. All steps are performed under air, and the conditions used for this catalyst preparation are given in Table 9.

TABLE 9
Conditions for the formaldehyde treatment of a Raney-type Ni catalyst, that avoids
the presence of dissolved Ni in the treatment solution.

| | | ml of a 5 wt- | | | | |
|---|---|---|---|---|---|---|
| | | % NaOH | | | | |
| | | aqueous | | | | |
| | | solution | | | | |
| | Grams | added to the | | mmol | | |
| | Catalyst (dry | catalyst | Formaldehyde | formaldehyde | gas above | Temperature |
| Catalyst N^{o} | basis) | suspension | (g) | per g cat | solution | (° C.) |
| E9a | 200 | 48 | 10.16 | 1.69 | air | RT |

RT = room temperature

### EXAMPLE 10

### Formaldehyde Treatment of a Raney-Type Ni Catalyst that Avoids the Presence of Dissolved Ni in the Treatment Solution

A Raney-type nickel catalyst with an average particle size of ˜53 μm is treated with formaldehyde according to the procedure described here so as to avoid the presence of dissolved Ni in the treatment suspension.

A 105 ml solution containing 42.35 grams of formaldehyde is added over 15 minutes to an aqueous suspension, containing ˜1350 grams of water and 500 grams (on a dry basis) of the catalyst at room temperature. During the addition of the formaldehyde solution, the pH of the catalyst suspension is maintained at 7 by the timely addition of a 5 wt % aqueous NaOH solution. The amount of the 5 wt % NaOH solution given to the slurry during the formaldehyde addition is totaled to 105 ml. The suspension of the treated catalyst was allowed to stir for an additional 45 minutes at room temperature. At the end of this 45 minutes, a metal analysis of the catalyst treatment suspension shows that the concentration of dissolved Ni was 0 ppm and further examination indicates, that no formaldehyde remained in the treatment solution. The catalyst is then allowed to settle to the bottom of the vessel. The overstanding solution is decanted, and the catalyst is washed twice with 1000 ml of water. Since the treatment solution does not contain dissolved Ni or formaldehyde, the washing step is optional. All steps are performed under air and the conditions used for this catalyst preparation are given in Table 10.

TABLE 10
Conditions for the formaldehyde treatment of a Raney-type Ni catalyst that avoids
the presence of dissolved Ni in the treatment solution.

| | | ml of a 5 wt- | | | | |
|---|---|---|---|---|---|---|
| | | % NaOH | | | | |
| | | aqueous | | | | |
| | | solution | | | | |
| | Grams | added to the | | mmol | | |
| | Catalyst (dry | catalyst | Formaldehyde | formaldehyde | gas above | Temperature |
| Catalyst N^{o} | basis) | suspension | (g) | per g cat | solution | (° C.) |
| E10a | 500 | 105 | 42.35 | 2.82 | air | RT |

RT = room temperature

### EXAMPLE 11

### Formaldehyde Treatment of a Raney-Type Ni Catalyst that Avoids the Presence of Dissolved Ni in the Treatment Solution

A Raney-type nickel catalyst with an average particle size of ˜53 μm is treated with formaldehyde according to the procedure described here so as to avoid the presence of dissolved Ni in the treatment suspension.

A 105 ml solution containing 42.35 grams of formaldehyde is mixed with 225 ml of a 5 wt.-% NaOH aqueous solution, and this 330 ml mixture is added over 20 minutes to an aqueous suspension, containing ˜1350 grams of water and 500 grams (on a dry basis) of the catalyst at room temperature. The suspension of the treated catalyst is allowed to stir for an additional hour at room temperature. At the end of this hour, a metal analysis of the catalyst treatment suspension shows, that the concentration of dissolved Ni is 0 ppm, and further examination indicates, that no formaldehyde remains in the treatment solution.

The catalyst is then allowed to settle to the bottom of the vessel. The overstanding solution is decanted, and the catalyst is washed twice with 1000 ml of water. Since the treatment solution does not contain dissolved Ni or formaldehyde, the washing step is optional. All steps are performed under air, and the conditions used for this catalyst preparation are given in Table 11.

TABLE 11
Conditions for the formaldehyde treatment of a Raney-type Ni catalyst that avoids
the presence of dissolved Ni in the treatment solution.

| | | ml of a 5 wt- | | | | |
|---|---|---|---|---|---|---|
| | | % NaOH | | | | |
| | | aqueous | | | | |
| | | solution | | | | |
| | Grams | added to the | | mmol | | |
| | Catalyst (dry | catalyst | Formaldehyde | formaldehyde | gas above | Temperature |
| Catalyst N^{o} | basis) | suspension | (g) | per g cat | solution | (° C.) |
| E11a | 500 | 225 | 42.35 | 2.82 | air | RT |

RT = room temperature

### EXAMPLE 12

### Treatment of a Raney-Type Ni Catalyst with Formaldehyde in the Presence of a Templating Agent (Sorbitol)

A commercially available Raney-type nickel catalyst with an average particle size of ˜53 μm is initially suspended in a aqueous solution containing a molecule, that would adsorb on the catalyst's surface thereby creating a template prior to the deposition of carbonaceous materials via formaldehyde treatment. In the beginning, 32.35 grams (on a dry basis) of the catalyst is suspending in 122 ml of a stirring 50 wt % sorbitol aqueous solution for 1 hour at room temperature. This is followed by the addition of 2.011 grams of a 37 wt % formaldehyde aqueous solution and the continued stirring of this solution for one hour at room temperature. All steps are performed under air. After treatment, the catalyst is allowed to settle. The overstanding aqueous solution is decanted, and the catalyst is washed three times with 400 ml of distilled water. Details of this treatment are given in table 12.

TABLE 12
Conditions for the formaldehyde treatment of a Raney-type Ni catalyst in the
presence of sorbitol.

| | Grams | aqueous | | mmol | | |
|---|---|---|---|---|---|---|
| | Catalyst (dry | Templating | Formaldehyde | formaldehyde | gas above | Temperature |
| Catalyst N^{o} | basis) | Solution | (g) | per g cat | solution | (° C.) |
| E12a | 32.35 | 50 wt % | 0.7441 | 0.77 | air | RT |
| | | Sorbitol | | | | |

RT = room temperature

### EXAMPLE 13

### Treatment of a Raney-Type Ni Catalyst with Formaldehyde in the Presence of a Templating Agent (Glucose)

A commercially available Raney-type nickel catalyst with an average particle size of ˜53 μm is initially suspended in a aqueous solution containing a molecule, that would adsorb on the catalyst's surface thereby creating a template prior to the deposition of carbonaceous materials via formaldehyde treatment.

In the beginning, 32.35 grams (on a dry basis) of the catalyst are suspending in 122 ml of a stirring 50 wt.-% glucose aqueous solution for 1 hour at room temperature. This is followed by the addition of 2.011 grams of a 37 wt % formaldehyde aqueous solution and the continued stirring of this solution for one hour at room temperature. All steps are performed under air. After treatment, the catalyst is allowed to settle. The overstanding aqueous solution is decanted, and the catalyst was washed three times with 400 ml of distilled water. Details of this treatment are given in table 13.

TABLE 13
Conditions for the formaldehyde treatment of a Raney-type Ni catalyst in the
presence of glucose.

| | | aqueous | | mmol | | |
|---|---|---|---|---|---|---|
| Catalyst | Grams Catalyst | Templating | Formaldehyde | formaldehyde | gas above | Temperature |
| N^{o} | (dry basis) | Solution | (g) | per g cat | solution | (° C.) |
| E13a | 32.35 | 50 wt % Glucose | 0.7441 | 0.77 | air | RT |

RT = room temperature

### EXAMPLE 14

### Treatment of a Raney-Type Ni Catalyst with Formaldehyde in the Presence of a Templating Agent (Mannitol)

A commercially available Raney-type nickel catalyst with an average particle size of ˜53 μm is initially suspended in a aqueous solution, containing a molecule, that would adsorb on the catalyst's surface, thereby creating a template prior to the deposition of carbonaceous materials via formaldehyde treatment. In the beginning, 32.35 grams (on a dry basis) of the catalyst is suspending in 122 ml of a stirring 10 wt.-% mannitol aqueous solution for 1 hour at room temperature. This is followed by the addition of 2.011 grams of a 37 wt % formaldehyde aqueous solution and the continued stirring of this solution for one hour at room temperature. All steps are performed under air. After treatment, the catalyst is allowed to settle. The overstanding aqueous solution is decanted, and the catalyst is washed three times with 400 ml of distilled water. Details of this treatment are given in table 14.

TABLE 14
Conditions for the formaldehyde treatment of a Raney-type Ni catalyst in the
presence of glucose.

| | | | | | gas | |
|---|---|---|---|---|---|---|
| Catalyst | Grams Catalyst | aqueous Templating | | mmol formaldehyde | above | Temperature |
| N^{o} | (dry basis) | Solution | Formaldehyde (g) | per g cat | solution | (° C.) |
| E14a | 32.35 | 10 wt % Mannitol | 0.7441 | 0.77 | air | RT |

RT = room temperature

### EXAMPLE 15

### Treatment of a Raney-Type Ni Catalyst with Formaldehyde in the Presence of a Templating Agent (Mannose)

A commercially available Raney-type nickel catalyst with an average particle size of ˜53 μm is initially suspended in a aqueous solution containing a molecule that would adsorb on the catalyst's surface thereby creating a template prior to the deposition of carbonaceous materials via formaldehyde treatment.

In the beginning, 32.35 grams (on a dry basis) of the catalyst is suspending in 122 ml of a stirring 20 wt. % mannose aqueous solution for 1 hour at room temperature. This is followed by the addition of 2.011 grams of a 37 wt.-% formaldehyde aqueous solution and the continued stirring of this solution for one hour at room temperature. All steps are performed under air. After treatment, the catalyst is allowed to settle. The overstanding aqueous solution is decanted, and the catalyst is washed three times with 400 ml of distilled water. Details of this treatment are given in table 15.

TABLE 15
Conditions for the formaldehyde treatment of a Raney-type Ni catalyst in the
presence of mannose.

| | | | | | gas | |
|---|---|---|---|---|---|---|
| Catalyst | Grams Catalyst | aqueous Templating | | mmol formaldehyde | above | Temperature |
| N^{o} | (dry basis) | Solution | Formaldehyde (g) | per g cat | solution | (° C.) |
| E15a | 32.35 | 20 wt % Mannose | 0.7441 | 0.77 | air | RT |

RT = room temperature

### EXAMPLE 16

### Treatment of a Raney-Type Cu Catalyst with Formaldehyde

A commercially available Raney-type copper catalyst with an average particle size of ˜43 μm is treated with different amounts of formaldehyde according to the procedure described here. The desired quantities of the catalyst are suspended in an aqueous solution containing formaldehyde at the desired temperature. This suspension is stirred for 1 hour at the desired temperature under air. After treatment, the catalyst is allowed to settle as it cooled to room temperature (if needed). The overstanding aqueous solution is decanted, and the catalyst is washed three times with distilled water. The specific conditions used for the preparation of each catalyst are given in detail in Table 1.

TABLE 16
Conditions for the formaldehyde treatment of a Raney-type Cu catalyst.

| | | Grams | | | | | | water for |
|---|---|---|---|---|---|---|---|---|
| | mmol | Catalyst | | aqueous | gas | | | each |
| Catalyst | formaldehyde | (dry | Formaldehyde | solution | above | Temperature | Time | washing |
| N^{o} | per g cat | basis) | (g) | (ml) | solution | (° C.) | (h) | step (ml) |
| E16a | 0.765 | 59.0 | 1.354 | 213 | air | RT | 1 | 472 |
| E16b | 0.765 | 59.0 | 1.354 | 213 | air | 80 | 1 | 472 |
| E16c | 0.765 | 29.5 | 0.6771 | 107 | air | RT | 1 | 236 |
| E16d | 0.498 | 29.5 | 0.4403 | 107 | air | RT | 1 | 236 |

RT = room temperature

### EXAMPLE 17

### Treatment of a Raney-Type Ni Catalyst with Formaldehyde in the Presence of a Templating Agent (Tartaric Acid with or without NaBr)

A commercially available Raney-type nickel catalyst (32.25 grams on a dry basis) with an average particle size of ˜53 μm is initially washed three times with 500 ml of distilled water and then stirred 5 minutes at room temperature in 500 ml of a solution containing 5 grams of the desired enantiomer of tartaric acid. For some of the catalysts, 25 grams of NaBr are also added to this solution. During this 5 minute tartaric acid treatment (with or without NaBr) the pH is kept between 3.1 and 3.3 by the addition of the required amount of a 10% wt NaOH solution. After the 5 minute tartaric acid treatment, 2.011 grams of a 37 wt.-% formaldehyde solution are added, and the resulting catalyst suspension is stirred for an additional hour. All steps are performed under air. After treatment, the catalyst is allowed to settle. The overstanding aqueous solution is decanted and the catalyst is washed three times with 400 ml of distilled water. In one of these examples, the treatment above is performed without tartaric acid and only in the presence of NaBr. Details of these treatments are in table 17.

TABLE 17
Conditions for the formaldehyde treatment of a Raney-type Ni catalyst in the
presence of tartaric acid with or without NaBr.

| | | | | | gas | |
|---|---|---|---|---|---|---|
| Catalyst | Grams Catalyst | aqueous Templating | | mmol formaldehyde | above | Temperature |
| N^{o} | (dry basis) | Solution | Formaldehyde (g) | per g cat | solution | (° C.) |
| E17a | 32.35 | L(+) Tartaric | 0.7441 | 0.767 | air | RT |
| | | Acid & NaBr | | | | |
| E17b | 32.35 | L(+) Tartaric | 0.7441 | 0.767 | air | RT |
| | | Acid | | | | |
| E17c | 32.35 | D(−) Tartaric | 0.7441 | 0.767 | air | RT |
| | | Acid & NaBr | | | | |
| E17d | 32.35 | D(−) Tartaric | 0.7441 | 0.767 | air | RT |
| | | Acid | | | | |
| E17e | 32.35 | NaBr | 0.7441 | 0.767 | air | RT |

RT = room temperature

### EXAMPLE 18

### Treatment of a Raney-Type Ni Catalyst with Formaldehyde Followed by Mo Doping

A commercially available Raney-type nickel catalyst with an average particle size of ˜53 μm is initially treated with formaldehyde followed by Mo doping via the deposition of an ammonium molybdate salt on the catalyst's surface as described here.

The catalyst (110 grams on a dry basis) is suspended in 400 ml of a stirred aqueous solution containing 3.887 grams of formaldehyde for 1 hour at room temperature under air. After the formaldehyde treatment, the catalyst is allowed to settle. The overstanding aqueous solution is decanted, and the catalyst is washed three times with 880 ml of distilled water.

The formaldehyde treated catalyst is then suspended in 200 ml of distilled water after which, enough ammonium molybdate salt was given to the catalyst to give it a final Mo content of 0.9 wt.-%. After adding the Mo, the resulting catalyst suspension is then stirred for an additional 2.5 hours. After treatment, the catalyst is allowed to settle. The overstanding aqueous solution was decanted, and the catalyst was washed twice with 200 ml of distilled water. The specific conditions used for the preparation of this catalyst are given in Table 18.

TABLE 18
Conditions for the formaldehyde treatment of a Raney-type Ni catalyst followed by
doping it with an ammonium molybdate salt.

| | mmol | | | aqueous | | | | | |
|---|---|---|---|---|---|---|---|---|---|
| Catalyst | formaldehyde | Grams Catalyst | Formaldehyde | solution | gas above | Temperature | Time | | Final |
| N^{o} | per g cat | (dry basis) | (g) | (ml) | solution | (° C.) | (h) | MO salt | wt-% Mo |
| E18a | 1.177 | 110 | 3.885 | 400 | air | RT | 1 | Ammonium | 0.9 |
| | | | | | | | | Molybdate | |

T = room temperature

### EXAMPLE 19

### Treatment of a Raney-Type Ni Catalyst with Sodium Formate Followed by Mo Doping

A commercially available Raney-type nickel catalyst with an average particle size of ˜53 μm is initially treated with sodium formate, followed by Mo doping via the deposition of a sodium molybdate salt on the catalyst's surface as described here.

The catalyst (110 grams on a dry basis) is suspended in 385 ml of a stirred aqueous solution, containing 9.16 grams of sodium formate for 1 hour at 90° C. under air. After the sodium formate treatment, the catalyst is allowed to settle while it cooled to room temperature. The overstanding aqueous solution is decanted, and the catalyst is washed three times with 1360 ml of distilled water.

The sodium formate treated catalyst is then suspended in 200 ml of distilled water after which, enough sodium molybdate salt is given to the catalyst to give it a final Mo content of 1.2 wt.-%. After adding the Mo, the resulting catalyst suspension is then stirred for an additional 2.5 hours. After treatment, the catalyst is allowed to settle. The overstanding aqueous solution is decanted, and the catalyst is washed twice with 200 ml of distilled water. The specific conditions used for the preparation of this catalyst are given in Table 19.

TABLE 19
Conditions for the formaldehyde treatment of a Raney-type Ni catalyst, followed by
doping it with a sodium molybdate salt.

| | mmol | Grams | | aqueous | gas | | | | |
|---|---|---|---|---|---|---|---|---|---|
| Catalyst | formaldehyde | Catalyst | Formaldehyde | solution | above | Temperature | Time | | Final wt- |
| N^{o} | per g cat | (dry basis) | (g) | (ml) | solution | (° C.) | (h) | MO salt | % Mo |
| E19a | 1.225 | 110 | 9.16 | 200 | air | 90° C. | 1 | Sodium | 1.2 |
| | | | | | | | | Molybdate | |

### EXAMPLE 20

### The Production of Raney-Type Ni Hollow Spheres Fixed Bed Catalysts and their Treatment with Formaldehyde

Activated Raney-type Ni hollow spheres are produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 50 wt.-% Ni/50 wt.-% Al alloy and Ni binder onto a fluidized bed of styrofoam balls.

This spraying was performed in 2 steps. After impregnation, the coated styrofoam spheres are first dried and then calcined at 700° C. to burn out the styrofoam and stabilize the metal shell. The hollow spheres of alloy are then activated in a 20 to 30% caustic solution from 1.5 to 2 hours at ˜80 to 100° C. The catalyst is then washed and stored in a mildly caustic aqueous solution (pH ˜10.5) before being modified.

The desired amount of the catalytic hollow spheres is placed into a metal basket that is then submerged for an hour into a stirred aqueous solution containing the appropriate amount of formaldehyde at room temperature. After this treatment, the modified catalyst is removed from the formaldehyde solution, washed 3 times with water and stored under water until use. Under the conditions used for the preparation of these catalysts, all of the end products had a bulk density of 1.05 grams per ml. The conditions of these formaldehye treatments are listed in table 20.

TABLE 20
Conditions for the formaldehyde treatment of the Raney-type Ni hollow spheres fixed
bed catalysts.

| | mmol | Grams | | aqueous | | | | |
|---|---|---|---|---|---|---|---|---|
| Catalyst | formaldehyde | Catalyst | Formaldehyde | solution | gas above | Temperature | Time | Bulk Density of the |
| N^{o} | per g cat | (dry basis) | (g) | (ml) | solution | (° C.) | (h) | Catalyst (g/ml) |
| E20a | 0.77 | 97 | 2.231 | 413 | air | RT | 1 | 1.05 |
| E20a | 1.30 | 97 | 3.785 | 413 | air | RT | 1 | 1.05 |

RT = room temperature

### EXAMPLE 21

### The Production of Raney-Type Ni Hollow Spheres Fixed Bed Catalysts and their Treatment with Sodium Formate

Activated Raney-type Ni hollow spheres are produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol, containing suspension of the 53 wt.-% Ni/47 wt.-% Al alloy and Ni binder onto a fluidized bed of styrofoam balls.

This spraying was performed in 2 steps.

After impregnation, the coated styrofoam spheres are first dried and then calcined at 750° C. to burn out the styrofoam and stabilize the metal shell. The hollow spheres of alloy are then activated in a 20 to 30% caustic solution from 1.5 to 2 hours at ˜80 to 100° C. The catalyst is then washed and stored in a mildly caustic aqueous solution (pH ˜10.5) before being modified. The desired amount of the catalytic hollow spheres is placed into a metal basket that is then submerged for an hour into a stirred aqueous solution containing the appropriate amount of sodium formate as described in table 21.

After this treatment, the modified catalyst is removed from the sodium formate solution, washed 3 times with water and stored under water until use. Under the conditions used for this catalyst, the end product has a bulk density of 0.907 grams per ml.

TABLE 21
Conditions for the sodium formate treatment of the Raney-type Ni hollow spheres
fixed bed catalysts.

| | | Grams | | | | | | |
|---|---|---|---|---|---|---|---|---|
| | mmol sodium | Catalyst | sodium | aqueous | | | | Bulk Density of |
| Catalyst | formate per | (dry | formate | solution | gas above | Temperature | Time | the Catalyst |
| N^{o} | g cat | basis) | (g) | (ml) | solution | (° C.) | (h) | (g/ml) |
| E21a | 1.30 | 97 | 8.579 | 423 | air | 105 | 1 | 0.907 |

RT = room temperature

### EXAMPLE 22

### The Production of Raney-Type Ni Hollow Spheres Fixed Bed Catalysts and their Treatment with Formaldehyde

Activated Raney-type Ni hollow spheres are produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 53 wt.-% Ni/47 wt.-% Al alloy and Ni binder onto a fluidized bed of styrofoam balls.

This spraying was performed in 2 steps.

After impregnation, the coated styrofoam spheres are first dried and then calcined at 750° C. to burn out the styrofoam and stabilize the metal shell. The hollow spheres of alloy are then activated in a 20 to 30% caustic solution from 1.5 to 2 hours at ˜80 to 100° C. The catalyst is then washed and stored in a mildly caustic aqueous solution (pH ˜10.5) before being modified. The desired amount of the catalytic hollow spheres is placed into a metal basket that is then submerged for an hour into a stirred aqueous solution containing the appropriate amount of formaldehyde at room temperature.

After this treatment, the modified catalyst is removed from the formaldehyde solution, washed 3 times with water and stored under water until use. Under the conditions used for the preparation of these catalysts, all of the end products has a bulk density of 0.907 grams per ml. The conditions of the formaldehye treatments are listed in table 22.

TABLE 22
Conditions for the formaldehyde treatment of the Raney-type Ni hollow spheres fixed
bed catalysts.

| | | Grams | | | | | | |
|---|---|---|---|---|---|---|---|---|
| | mmol | Catalyst | | aqueous | | | | Bulk Density of |
| Catalyst | formaldehyde | (dry | Formaldehyde | solution | gas above | Temperature | Time | the Catalyst |
| N^{o} | per g cat | basis) | (g) | (ml) | solution | (° C.) | (h) | (g/ml) |
| E22a | 0.77 | 97 | 2.231 | 413 | air | RT | 1 | 0.907 |
| E22b | 1.30 | 97 | 3.785 | 413 | air | RT | 1 | 0.907 |

RT = room temperature

### COMPARATIVE EXAMPLE 1

### Raney-Type Ni Catalyst

A commercially available Raney-type nickel catalyst with an average particle size of ˜53 μm (CE1) is used in comparison to those modified according to this invention.

### COMPARATIVE EXAMPLE 2

### Raney-Type Ni Catalyst

A commercially available Raney-type nickel catalyst with an average particle size of ˜28 μm (CE2) is used in comparison to those modified according to this invention.

### COMPARATIVE EXAMPLE 3

### Raney-Type Ni Catalyst, Whose Precursor Alloy Contains Mo

A commercially available Raney-type nickel catalyst, whose precursor alloy contains Mo (CE3), is used in comparison to those modified according to this invention.

### COMPARATIVE EXAMPLE 4

### Raney-Type Ni Catalyst, that is Post-Activation Doped with Mo

A Raney-type Ni catalyst, that is post-activation doped with Mo (via an ammonium molybdate compound) to a level of 1.2% Mo and has an average particle size of ˜28 μm (CE4) is used in comparison to those modified according to this invention.

### COMPARATIVE EXAMPLE 5

### Raney-Type Ni Catalysts Modified with Formaldehyde According to Deglisher et al U.S. Pat. No. 6,521,564.

An aqueous solution containing 8.47 grams of formaldehyde is added under nitrogen at 25° C. to an aqueous suspension of 100 grams of a Raney type Ni catalyst having an average particle size of ˜53 μm. The treated suspension is then stirred for one hour at room temperature under nitrogen. The resulting catalyst suspension contains more than 500 ppm Ni and more than 100 ppm formaldehyde in the over standing solution and even after washing the catalyst. The catalyst suspension still contains a considerable amount of dissolved Ni, thereby creating wash water disposal problems and problems with the use of this catalyst (CE5). Since formaldehyde came off during the treatment, it is not possible to determine how much of the desired 2.82 mmole of formaldehyde per gram of catalyst is retained.

### COMPARATIVE EXAMPLE 6

### Raney-Type Ni Catalysts, Modified with Formaldehyde According to Deglisher et al U.S. Pat. No. 6,521,564.

An aqueous solution containing 44.36 grams of formaldehyde is added under nitrogen at 25° C. to an aqueous suspension of 500 grams of a Raney type Ni catalyst having an Average particle size of ˜53 μm. The treated suspension is then stirred for one hour at room temperature under nitrogen. The resulting catalyst suspension contains more than 500 ppm Ni and more than 100 ppm formaldehyde in the over standing solution and even after washing the catalyst, the catalyst suspension still contains a considerable amount of dissolved Ni, thereby creating wash water disposal problems and problems with the use of this catalyst (CE6). Since formaldehyde came off during the treatment, it is not possible to determine how much of the desired 2.96 mmole of formaldehyde per gram of catalyst is retained.

### COMPARATIVE EXAMPLE 7

### Raney-Type Cu Catalyst

A commercially available Raney-type copper catalyst with an average particle size of ˜43 μm (CE7) is used in comparison to those modified according to this invention.

### COMPARATIVE EXAMPLE 8

### Raney-Type Ni Catalyst that is Post Activation Doped with Mo

A Raney-type Ni catalyst, that is post activation doped with Mo (via a sodium molybdate compound) to a level of 0.9% Mo and has an average particle size of ˜53 μm (CE8), is used in comparison to those modified according to this invention.

### COMPARATIVE EXAMPLE 9

### Raney-Type Ni Catalyst, that is Post Activation Doped with Mo

A Raney-type Ni catalyst, that is post activation doped with Mo (via a sodium molybdate compound) to a level of 1.2% Mo and has an average particle size of ˜53 μm (CE9), is used in comparison to those modified according to this invention.

### COMPARATIVE EXAMPLE 10

### The Production of Raney-Type Ni Hollow Spheres Fixed Bed Catalysts

Activated Raney-type Ni hollow spheres are produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 50 wt. % Ni/50 wt. % Al alloy and Ni binder onto a fluidized bed of styrofoam balls.

This spraying was performed in 2 steps.

After impregnation, the coated styrofoam spheres are first dried and then calcined at 700° C. to burn out the styrofoam and stabilize the metal shell. The hollow spheres of alloy are then activated in a 20 to 30% caustic solution from 1.5 to 2 hours at ˜80 to 100° C. The catalyst (CE10) is then washed and stored in a mildly caustic aqueous solution (pH ˜10.5). The end product has a bulk density of 1.05 grams per ml.

### EXAMPLE 11

### The Production of Raney-Type Ni Hollow Spheres Fixed Bed Catalysts

Activated Raney-type Ni hollow spheres are produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the 53 wt.-% Ni/47 wt.-% Al alloy and Ni binder onto a fluidized bed of styrofoam balls. This spraying is performed in 2 steps. After impregnation, the coated styrofoam spheres are first dried and then calcined at 750° C. to burn out the styrofoam and stabilize the metal shell. The hollow spheres of alloy are then activated in a 20 to 30% caustic solution from 1.5 to 2 hours at ˜80 to 100° C. The catalyst (CE11) is then washed and stored in a mildly caustic aqueous solution (pH ˜10.5). The end product had a bulk density of 0.907 grams per ml.

### COMPARATIVE EXAMPLE 12

### A Raney-Type Ni Catalyst, that is Modified with the Enantiomers of Tartaric Acid and Sodium Bromide

A commercially available tartaric acid/sodium bromide modified Raney-type nickel catalyst with an average particle size of ˜53 μm is used for comparison to those similarly modified according to this invention. This modification is either carried out with L(+) or D(−) tartaric acid as shown in table EC12 below.

TABLE 23
The characteristic of the tartaric acid and
sodium bromide modified Raney-type Ni catalysts.

| | Enantiomer of | Presence of NaBr |
|---|---|---|
| Catalyst N^{o} | tartaric acid | (Y or N) |
| CE12a | L (8+) | Y |
| CE12b | D (−) | Y |

### APPLICATION EXAMPLE 1

### Benzonitrile Hydrogenation

The hydrogenation of benzonitrile (BN) is carried out in a 1 liter steel autoclave with 1.3 grams of catalyst (on a dry basis), 32.5 grams of benzonitrile and 514.15 grams of Methanol as solvent. The reactor is initially purged three times with nitrogen followed by three purges with hydrogen before the hydrogen pressure is set at 38 bar while stirring the reaction mixture at 2000 rpm. The temperature is then ramped from room temperature to 105° C. over 65 minutes and once hydrogen consumption started, the hydrogen pressure is kept constant at 41 bar. Some reactions are also carried out in the presence of measured amounts of an aqueous 32% ammonia solution. At the end of the reaction, the autoclave is cooled down and the reaction mixture is analyzed by gas chromatography (GC). During this test, the starting reaction temperature and the time to reach 98% hydrogen consumption are listed as indications of catalyst activity. The GC analysis provides the percent conversion (% Con.) and the percent selectivities of benzylamine (BA), dibenzylimine (DBI) and dibenzylamine (DBA). The results of these tests are listed in table 24.

TABLE 24
The results of the benzonitrile hydrogenation
tests.

| | | Starting | Time for | | | | |
|---|---|---|---|---|---|---|---|
| | ml of 32% | Reaction | 98% H2 | | | | |
| | liquid | Temp., | uptake. | % | % Yield | | |
| Catalyst | Ammonia | ° C. | min. | Conv. | BA | DBA | DBI |
| CE1 | 0 | 60 | 21 | 100 | 64 | 36 | 0 |
| CE1 | 1 | 57 | 21 | 100 | 64 | 36 | 0 |
| CE1 | 3 | 55 | 20 | 100 | 68 | 32 | 0 |
| CE1 | 5 | 57 | 18 | 100 | 72 | 28 | 0 |
| CE1 | 10 | 60 | 19 | 100 | 80 | 20 | 0 |
| CE3 | 0 | 52 | 15 | 100 | 56 | 44 | 0 |
| CE3 | 10 | 40 | 15 | 100 | 75 | 25 | 0 |
| E1c | 0 | 68 | 28 | 100 | 72 | 28 | 0 |
| E1d | 0 | 69 | 25 | 100 | 72 | 28 | 0 |
| E1f | 0 | 69 | 27 | 100 | 73 | 25 | 2 |
| E1g | 0 | 70 | 27 | 100 | 73 | 25 | 2 |
| E1h | 0 | 71 | 26 | 100 | 74 | 21 | 5 |
| E1i | 0 | 73 | 28 | 100 | 75 | 21 | 4 |
| E1j | 0 | 79 | 30 | 100 | 73 | 15 | 12 |
| E1k | 0 | 76 | 32 | 100 | 72 | 13 | 15 |
| E2a | 0 | 52 | 14 | 100 | 61 | 39 | 0 |
| E2b | 0 | 59 | 16 | 100 | 65 | 35 | 0 |
| E2d | 0 | 62 | 20 | 100 | 73 | 27 | 0 |
| E2e | 0 | 64 | 19 | 100 | 78 | 22 | 0 |
| E2f | 0 | 67 | 21 | 100 | 80 | 19 | 1 |
| E2g | 0 | 71 | 24 | 100 | 79 | 16 | 5 |
| E2h | 0 | 65 | 25 | 100 | 77 | 14 | 9 |
| E2i | 0 | 66 | 28 | 100 | 75 | 10 | 15 |
| E2j | 0 | 68 | 33 | 100 | 71 | 8 | 21 |
| E2k | 0 | 72 | 33 | 100 | 67 | 7 | 26 |
| E3a | 0 | 68 | 25 | 100 | 74 | 22 | 4 |
| E3b | 0 | 74 | 19 | 100 | 72 | 12 | 16 |
| E3c | 0 | 83 | 43 | 100 | 68 | 7 | 25 |
| E4a | 0 | 59 | 19 | 100 | 70 | 30 | 0 |
| E4b | 0 | 60 | 23 | 100 | 72 | 25 | 3 |
| E4i | 0 | 67 | 21 | 100 | 77 | 23 | 0 |
| E4d | 0 | 63 | 21 | 100 | 78 | 21 | 1 |
| E4e | 0 | 65 | 21 | 100 | 76 | 21 | 3 |
| E4f | 0 | 66 | 20 | 100 | 77 | 19 | 4 |
| E4g | 0 | 66 | 22 | 100 | 77 | 19 | 4 |
| E4l | 0 | 67 | 24 | 100 | 78 | 22 | 0 |
| E4j | 0 | 67 | 25 | 100 | 81 | 18 | 1 |
| E4m | 0 | 69 | 22 | 100 | 74 | 12 | 14 |
| E4k | 0 | 69 | 23 | 100 | 80 | 19 | 1 |
| E4h | 0 | 66 | 20 | 100 | 79 | 21 | 0 |
| E4c | 0 | 65 | 21 | 100 | 81 | 18 | 1 |
| E1f | 10 | 70 | 24 | 100 | 91 | 9 | 0 |
| E2f | 5 | 65 | 23 | 100 | 89 | 11 | 0 |
| E2f | 10 | 64 | 20 | 100 | 99 | 1 | 0 |
| E4k | 10 | 67 | 22 | 100 | 99 | 1 | 0 |
| CE2 | 0 | 65 | 18 | 100 | 62 | 38 | 0 |
| CE4 | 0 | 55 | 22 | 100 | 55 | 45 | 0 |
| CE9 | 0 | 56 | 25 | 100 | 55 | 46 | 0 |
| E19a | 0 | 79 | 56 | 100 | 65 | 29 | 6 |
| E18a | 0 | 67 | 25 | 100 | 81 | 18 | 1 |

### APPLICATION EXAMPLE 2

### The Slurry Phase Hydrogenation of a Tallow Nitrile Mixture

The slurry phase hydrogenation of a tallow nitrile mixture consisting predominantly of C₁₆ and C₁₈ with a small amount of C₁₄, C₂₀ and other long chain aliphatic fatty nitriles with a iodine value (IV) of ˜51 is carried out in a 1 liter steel autoclave. Initially the autoclave is charged with 1 gram of catalyst (on a dry basis) and 500 grams of the above mentioned tallow nitrile followed by three purges with nitrogen and then three purges with ammonia. The reagent is then saturated with 6 bars of ammonia while stirring the mixture at 2000 rpm followed by ramping from room temperature to 140° C. in about 90 minutes. After reaching 140° C., the stirring is stopped, the ammonia pressure is adjusted to the desired value (10, 16.5 or 20 bar), hydrogen is added to bring the pressure to 40 bars and the reaction is started again by stirring at 2000 rpm. Samples are taken from the reaction mixture after the consumption of every 20 liters of hydrogen or every hour as determined by which occurs first. A sample is also taken at the end of the reaction. The iodine value (IV), secondary and tertiary amine value (2/3A) and the total amine value (TAV) are all determined for the fresh tallow nitrile and the hydrogenation samples. The IV is determined by a modified Wijs method similar to method Tg 1-64 of the American Oil Chemists' Society (AOCS) where the only difference is the use of cyclohexane instead of carbon tetrachloride. The 2/3A value is determined by the official AOCS method Tf 2a-64 and the TAV is measured via the AOCS potenziometric titration method Tf 1a-64. The results of these tests are listed in tables 25 and 26.

TABLE 25
The results of the slurry phase tallow nitrile
hydrogenation tests.

| | | | H₂ | | | | |
|---|---|---|---|---|---|---|---|
| | NH₃ | Time | uptake | | | | % IV |
| Catalyst | (bar) | (min) | (nL) | TAV | ⅔ A | IV | Retention |
| CE2 | 20 | 0 | 0 | — | — | 51.4 | 100 |
| | | 30 | 20.0 | 57.7 | 1.3 | 50.2 | 98 |
| | | 75 | 44.1 | 118.3 | 1.7 | 50.1 | 97 |
| | | 120 | 59.5 | 154.6 | 2.1 | 50.2 | 98 |
| | | 180 | 71.5 | 188.6 | 2.4 | 48.9 | 95 |
| | | 245 | 77.8 | 205.0 | 2.4 | 47.9 | 93 |
| CE2 | 16.5 | 0 | 0 | — | — | 51.4 | 100 |
| | | 30 | 28.6 | 50.0 | 1.2 | 50.0 | 50 |
| | | 60 | 53.6 | 118.2 | 2.12 | 48.0 | 48 |
| | | 75 | 64.3 | 136.0 | 3.2 | 49.0 | 49 |
| | | 95 | 75.5 | 163.9 | 5.0 | 48.3 | 48 |
| | | 120 | 86.0 | 190.3 | 5.9 | 45.3 | 45 |
| | | 155 | 94.4 | 202.1 | 5.9 | 42.7 | 43 |
| | | 360 | 107.7 | 212.8 | 6.5 | 23.0 | 23 |
| CE2 | 10 | 0 | 0 | — | — | 51.1 | 100 |
| | | 19 | 20.0 | 50.8 | 1.4 | 49.9 | 98 |
| | | 36 | 40.0 | 97.5 | 3.1 | 49.5 | 97 |
| | | 55 | 60.0 | 139.7 | 4.6 | 47.8 | 94 |
| | | 85 | 80.0 | 183.3 | 7.1 | 44.6 | 87 |
| | | 114 | 90.0 | 202.4 | 8.4 | 40.8 | 80 |
| | | 267 | 100.7 | 213.1 | 9.3 | 23.7 | 46 |
| E5a | 16.5 | 0 | 0 | — | — | 51.4 | 100 |
| | | 45 | 20.9 | 53.6 | 1.6 | 51.2 | 100 |
| | | 83 | 40.0 | 98.3 | 2.4 | 51.5 | 100 |
| | | 132 | 60.0 | 148.3 | 3.5 | 51.5 | 100 |
| | | 186 | 75.4 | 187.0 | 3.90 | 50.7 | 99 |
| | | 253 | 84.7 | 206.0 | 4.0 | 49.1 | 96 |
| | | 310 | 87.1 | 216.0 | 4.02 | 47.5 | 92 |
| E5a | 10 | 0 | 0 | — | — | 49.5 | 100 |
| | | 36 | 20.0 | 49.4 | 0.8 | 49.7 | 101 |
| | | 67 | 40.0 | 105.2 | 0.8 | 50.7 | 102 |
| | | 103 | 60.0 | 149.6 | 3.8 | 50.4 | 102 |
| | | 156 | 80.0 | 199.3 | 5.8 | 48.4 | 98 |
| | | 300 | 88.8 | 215.6 | 6.7 | 41.7 | 84 |
| E6a | 10 | 0 | 0.0 | — | — | 50.4 | 100 |
| | | 17 | 20.0 | 41.0 | 1.6 | 49.8 | 99 |
| | | 32 | 40.0 | 92.7 | 2.6 | 49.2 | 98 |
| | | 50 | 60.0 | 140.1 | 4.0 | 48.6 | 97 |
| | | 78 | 80.0 | 186.5 | 6.0 | 46.1 | 92 |
| | | 300 | 97.0 | 211.2 | 8.0 | 26.1 | 52 |
| E6b | 10 | 0 | 0 | — | — | 50.1 | 100 |
| | | 27 | 20 | 35.4 | nd | 49.7 | 99 |
| | | 51 | 40 | 89.8 | 2.1 | 49.9 | 100 |
| | | 77 | 60 | 133.0 | 3.9 | 49.4 | 99 |
| | | 111 | 80 | 174.5 | 5.93 | 47.1 | 94 |
| | | 137 | 90 | 196.6 | 7.11 | 45.5 | 91 |
| | | 197 | 100 | 211.0 | 8.6 | 39.4 | 79 |
| | | 365 | 110.7 | 213.8 | 9.5 | 27.2 | 54 |
| | | 433 | 114.1 | 218.4 | 9.4 | 21.7 | 43 |
| CE1 | 10 | 0 | 0 | — | — | 51.0 | 100 |
| | | 27 | 20.0 | 46.4 | 1.8 | 50.0 | 98 |
| | | 55 | 40.0 | 88.0 | 3.5 | 48.6 | 95 |
| | | 88 | 60.9 | 133.8 | 5.4 | 46.9 | 92 |
| | | 129 | 80.0 | 175.4 | 7.6 | 43.8 | 86 |
| | | 169 | 91.0 | 196.8 | 8.5 | 40.4 | 79 |
| | | 238 | 98.1 | 207.3 | 9.8 | 33.2 | 65 |
| | | 316 | 101.8 | 211.6 | 9.7 | 26.0 | 51 |
| CE1 | 20 | 0 | 0 | — | — | 51.4 | 100 |
| | | 40 | 23.7 | 62.1 | 1.8 | 51.6 | 100 |
| | | 85 | 44.9 | 114.5 | 1.6 | 51.0 | 99 |
| | | 115 | 55.2 | 144.1 | 2.0 | 50.3 | 98 |
| | | 155 | 69.1 | 184.2 | 2.8 | 47.3 | 92 |
| | | 235 | 77.8 | 202.2 | 2.9 | 47.6 | 93 |
| | | 280 | 79.9 | 209.0 | 3.1 | 47.0 | 91 |
| E1f | 20 | 0 | 0 | — | — | 51.7 | 100 |
| | | 45 | 21.1 | 53.9 | 1.4 | 50.3 | 97 |
| | | 90 | 39.8 | 100.0 | 2.1 | 51.5 | 100 |
| | | 150 | 57.9 | 152.0 | 2.7 | 50.8 | 98 |
| | | 195 | 67.4 | 175.5 | 2.7 | 47.9 | 93 |
| | | 230 | 72.5 | 189.4 | 2.7 | 48.8 | 94 |
| E3e | 20 | 0 | 0 | — | — | 51.0 | 100 |
| | | 45 | 29.9 | nd | 0.9 | 51 | 100 |
| | | 90 | 52.2 | 133.6 | 1.7 | 51.0 | 100 |
| | | 120 | 62.9 | 164.2 | 2.1 | 51.0 | 100 |
| | | 280 | 80.8 | 219.4 | 2.9 | 47.0 | 92 |
| E3a | 10 | 0 | 0 | — | — | 49.9 | 100 |
| | | 49 | 20.0 | 64.5 | 1.0 | 49.7 | 100 |
| | | 192.8 | 40.0 | 83.2 | 2.6 | 49.8 | 100 |
| | | 154.8 | 60.2 | 129.0 | 4.5 | 48.2 | 97 |
| | | 234.0 | 80.0 | 167.6 | 6.5 | 46.8 | 94 |
| | | 293.9 | 90.0 | 194.5 | 7.7 | 45.2 | 91 |
| | | 430.0 | 101.1 | 208.5 | 8.7 | 39.5 | 79 |
| CE3 | 10 | 0 | 0 | — | — | 50.1 | 100 |
| | | 25 | 20.0 | 43.9 | 1.3 | 48.1 | 96 |
| | | 51 | 40.0 | 84.4 | 4.6 | 47.8 | 95 |
| | | 81 | 60.0 | 123.2 | 5.3 | 45.6 | 91 |
| | | 121 | 80.0 | 165.6 | 6.8 | 43.3 | 86 |
| | | 148 | 90.0 | 182.8 | 7.9 | 41.2 | 82 |
| | | 190 | 100.0 | 200.4 | 8.6 | 37.3 | 74 |
| | | 282 | 110.0 | 209.9 | 10.2 | 29.2 | 58 |
| | | 361 | 114.1 | 206.9 | 11.6 | 23.6 | 47 |
| CE3 | 20 | 0 | 0 | — | — | 51.4 | 100 |
| | | 24 | 20.0 | 57.3 | 1.6 | 51.1 | 99 |
| | | 49 | 40.0 | 108.5 | 2.2 | 50.7 | 99 |
| | | 80 | 60. | 164.3 | 2.4 | 49.3 | 96 |
| | | 141 | 76.5 | 209.6 | 3.7 | 45.0 | 88 |
| | | 200 | 79.2 | 218.3 | 3.8 | 36.9 | 72 |
| | | 242 | 80.1 | 215.5 | 3.9 | 37.2 | 72 |
| E2a | 20 | 0 | 0 | — | — | 52.0 | 100 |
| | | 17 | 20.0 | 58.4 | 1.1 | 52.0 | 100 |
| | | 35 | 40.0 | 103.5 | 0.8 | 51.6 | 99 |
| | | 57 | 60.0 | 149.3 | 1.9 | 50.4 | 97 |
| | | 92 | 80.0 | 195.8 | 2.4 | 49.3 | 95 |
| | | 149 | 87.8 | 212.2 | 2.7 | 45.1 | 87 |
| | | 210 | 89.6 | 211.2 | 2.8 | 40.7 | 78 |
| | | 273 | 91.5 | 210.8 | 2.8 | 35.6 | 69 |
| E2b | 20 | 0 | 0 | — | — | 54.4 | 100 |
| | | 33 | 20.0 | 54.1 | 0.8 | 53.7 | 99 |
| | | 65 | 40.0 | 86.6 | 1.1 | 54.2 | 100 |
| | | 108 | 60.0 | 146.9 | 2.2 | 53.5 | 98 |
| | | 171 | 77.0 | 177.8 | 2.3 | 50.0 | 92 |
| | | 227 | 83.7 | 191.7 | 2.5 | 49.3 | 91 |
| | | 289 | 85.3 | 198.6 | 2.5 | 47.2 | 87 |
| E2d | 20 | 0 | 0 | — | — | 51.2 | 100 |
| | | 30 | 20.0 | 51.1 | 0.8 | 51.6 | 101 |
| | | 64 | 40.0 | 99.1 | 1.3 | 51.5 | 101 |
| | | 109 | 60.0 | 149.7 | 1.9 | 50.5 | 99 |
| | | 182 | 79.1 | 195.0 | 2.1 | 49.4 | 96 |
| | | 238 | 86.0 | 210.0 | 2.2 | 48.2 | 94 |
| | | 317 | 88.8 | 217.4 | 2.5 | 45.3 | 88 |
| CE4 | 10 | 0 | 0 | — | — | 49.3 | 100 |
| | | 30 | 20.0 | 39.4 | 0.9 | 49.2 | 100 |
| | | 97 | 60.0 | 139.7 | 4.0 | 47.4 | 96 |
| | | 146 | 80.0 | 182.4 | 6.0 | 44.1 | 90 |
| | | 187 | 90.0 | 202.6 | 7.2 | 40.4 | 82 |
| | | 313 | 100.0 | 214.4 | 8.4 | 27.5 | 56 |
| | | 360 | 102.2 | 213.5 | 8.6 | 22.8 | 46 |
| E7a | 20 | 0 | 0 | — | — | 49.5 | 100 |
| | | 29 | 20.0 | 40.2 | 2.3 | 49.1 | 99 |
| | | 53 | 40.0 | 87.4 | 3.2 | 49.1 | 99 |
| | | 81 | 60.0 | 139.0 | 4.9 | 47.7 | 96 |
| | | 120 | 80.0 | 181.1 | 6.0 | 44.8 | 91 |
| | | 160 | 90.0 | 206.5 | 7.2 | 40.5 | 82 |
| | | 359 | 98.3 | 211.6 | 8.0 | 21.7 | 44 |

TABLE 26
A quick summary of the results from the slurry
phase tallow nitrile hydrogenation tests.

| | | | IV at | ⅔ amines |
|---|---|---|---|---|
| | | Time for | TAV = 200 | at TAV = 200 |
| | | TAV = 200 | (% IV | (mg KOH/g |
| Catalyst | pNH₃ (bar) | (min) | Retention) | sample) |
| CE2 | 10 | 110 | 80 | 8 |
| E6a | 10 | 110-120 | 85 | 7 |
| E6b | 10 | 140 | 90 | 7 |
| E5a | 10 | 160 | 95 | 6 |
| CE3 | 20 | 120 | 90 | 3 |
| E2a | 20 | 100 | 95 | 2.5 |
| E2d | 20 | 200 | 95 | 2 |

### APPLICATION EXAMPLE 3

### The Fixed Bed Hydrogenation of a Tallow Nitrile Mixture

The fixed bed hydrogenation of a tallow nitrile mixture consisting predominantly of C₁₆ and C₁₈ with a small amount of C₁₄, C₂₀ and other long chain aliphatic fatty nitrites with an iodine value (IV) of ˜51 is carried out with a tube reactor in the trickle phase over 60 ml of catalyst at the pressure of 60 bars with a fourfold excess of hydrogen with respect to the total saturation of the tallow nitrile mixture. The reaction is carried out at the temperature of 140° C. with the LHSV sequence of 2, 1 and 0.5 h-1 or 1 and 0.5 h-1. Two or three samples are collected for every LHSV. The iodine value (IV), secondary and tertiary amine value (2/3A) and the total amine value (TAV) are all determined for the fresh tallow nitrile and the hydrogenation samples. The IV is determined by a modified Wijs method similar to method Tg 1-64 of the American Oil Chemists' Society (AOCS) where the only difference is the use of cyclohexane instead of carbon tetrachloride. The 2/3A value is determined by the official AOCS method Tf 2a-64 and the TAV is measured via the AOCS potenziometric titration method Tf 1a-64. The results of these tests are listed in table 27.

TABLE 27
The results from the fixed bed trickle phase
tallow nitrile hydrogenation tests.

| | LHSV | Temperature | | | |
|---|---|---|---|---|---|
| Catalyst | (h⁻¹) | (° C.) | TAV | ⅔ A | IV |
| E22b | 2 | 140 | 24 | 3.7 | 51 |
| E21a | 1 | 140 | 59.2 | 13.8 | 44.8 |
| | 0.5 | 140 | 119.0 | 33.7 | 28.6 |
| CE11 | 2 | 140 | 47.5 | 13.5 | 46.5 |
| | 1 | 140 | 66 | 22.7 | 40.8 |
| | 0.5 | 140 | 123 | 46 | 24.6 |

### APPLICATION EXAMPLE 4

### The Slurry Phase Hydrogenation of Adiponitrile

The slurry phase hydrogenation of adiponitrile is carried out in a one liter steel autoclave with 3 grams of catalyst (on a dry basis), 86.4 grams of adiponitrile, 314 grams of ethanol and 20 grams of water. After purging the reactor three times with nitrogen and three times with hydrogen, the autoclave is pressurized to 25 bar and stirred at 2000 rpm before starting the temperature ramp from room temperature to 75° C. over about 60 minutes. Once the reaction started, the reaction pressure is kept constant at 25 bar. After the reaction is stopped, the reaction mixture is separated from the catalyst and analyzed by GC. The results are listed in table 28.

TABLE 28
The results from the slurry phase hydrogenation
of adiponitrile.

| | Starting | H₂ | Conver- | | | | |
|---|---|---|---|---|---|---|---|
| | Temperature | uptake | sion | Selectivity (%) | | | |
| Catalyst | (° C.) | (liter) | (%) | HMI | HMDA | ACN | Other |
| CE1 | <15 | 18.5 | 45 | 4.0 | 15.8 | 79.9 | 0.5 |
| E1c | 50 | 8.2 | 25 | 2.5 | 7.5 | 89.8 | 0.2 |
| E1f | 59 | 2.7 | 16 | 1.1 | 2.4 | 96.6 | 0.0 |

### APPLICATION EXAMPLE 5

### The Slurry Phase Hydrogenation of Fructose

Fructose is hydrogenated as 500 grams of its 40% aqueous solution at 50 bar in a 1 liter autoclave. The reaction temperature of 100° C. and 2.4 wt. % catalyst is used for Ni and 110° C. and 7.2 wt. % catalyst is used for Cu. The autoclave is initially charged with the catalyst and fructose solution followed by three purges with nitrogen and 4 purges with 5 bars of hydrogen. The reactor is then pressurized to 45 bars and agitation is started at 1015 rpm as the reaction mixture is heated from room temperature to the desired final reaction temperature. As reaction mixture is heated pressure built up due to the increased water vapor and once this pressure dropped due to the initial hydrogen consumption, the hydrogen pressure is then adjusted to 50 bars for the duration of the reaction. Samples are taken as reaction progressed and these are analyzed via HPLC. The results of these tests are listed in table 29.

TABEL 29
The results from the slurry phase hydrogenation of fructose.

| | | Start | | Reaction | |
|---|---|---|---|---|---|
| | Max activity | Temp., | Mannitol % | Time, | Final % |
| Catalyst | MI H₂/min | ° C. | Selectivity | min. | Conversion |
| CE1 | 1203 | 53.8 | 51.28 | 46 | 96.39 |
| E1b | 945 | 60.5 | 53.98 | 57 | 97.93 |
| E1c | 748 | 64.13 | 56.05 | 106 | 97.02 |
| E1e | 598 | 70.3 | 55.48 | 108 | 98.79 |
| E1f | 358 | 74.8 | 54.39 | 206 | 97.75 |
| E1h | 259 | 78.3 | 53.97 | 259 | 98.26 |
| E1k | 204 | 83.5 | 54.52 | 314 | 99.15 |
| E8a | 261 | 78.3 | 54.49 | 373 | 94.42 |
| E9a | 455 | 68.1 | 54.61 | 188 | 99.45 |
| CE5 | 148 | 83.5 | 51.96 | 424 | 90.35 |
| CE3 | 1503 | 37.5 | 43.99 | 50 | 99.53 |
| E2c | 1236 | 51.1 | 51.49 | 40 | 96.01 |
| E2d | 885 | 58.3 | 51.06 | 57 | 98.29 |
| E2e | 664 | 66.5 | 50.72 | 82 | 96.81 |
| CE7 | nd | 85.5 | 64.17 | 347 | 92.69 |
| E16b | 165 | 99.9 | 67.7 | 446 | 92.13 |
| E16c | 291 | 92 | 64.96 | 343 | 92.80 |
| E16d | 329 | 86.3 | 66.23 | 303 | 94.49 |
| E12a | 697 | 66.7 | 54.79 | 108 | 98.33 |
| E13a | 427 | 71.3 | 53.22 | 153 | 98.81 |
| CE12a | 375 | 73.7 | 44.83 | 224 | 99.18 |
| E17a | 475 | 68 | 50.67 | 135 | 99.19 |
| CE12b | 359 | 72.7 | 47.32 | 212 | 98.73 |
| E17b | 448 | 68 | 50.32 | 158 | 99.09 |
| E17c | 461 | 69.5 | 51.02 | 120 | 98.88 |
| E17d | 513 | 68.6 | 53.05 | 129 | 98.89 |
| E17e | 736 | 59.4 | 52.1 | 102 | 98.99 |

### APPLICATION EXAMPLE 6

### The Fixed Bed Trickle Phase Hydrogenation of Fructose

A 40 wt. % fructose solution is hydrogenated over 60 ml of catalyst in the trickle phase under 80 bars of hydrogen with a tubular fixed bed reactor. There is a 20 fold excess of hydrogen and the liquid hour space velocities (LHSV) used for these tests were 0.2, 0.3, 0.4 and 0.5 h-1. The reaction temperatures of 90, 100, 110 and 120° C. are used for each of the above mentioned LHSV. The product mixture is analyzed by HPLC and the results are listed in table 30. From the results presented here, one can see that the controlled deposition of carbonaceous species on the catalyst led to an increase in the mannitol selectivity and a fitting of the catalyst's activity to the mass transfer limitations of the reactor under the conditions used.

TABLE 30
The results of the fixed bed trickle phase
hydrogenation of fructose.

| | Time | | Temperature | | % Selectivity |
|---|---|---|---|---|---|
| Catalyst | (h) | LHSV [h⁻¹] | ° C. | Conversion | Mannitol |
| CE10 | 3 | 0.2 | 90 | 74.84 | 49.76 |
| | 5.33 | 0.3 | 90 | 51.66 | 49.29 |
| | 7.33 | 0.4 | 90 | 40.09 | 49.54 |
| | 9.13 | 0.5 | 90 | 32.3 | 49.69 |
| | 3 | 0.2 | 100 | 76.4 | 48.56 |
| | 5.33 | 0.3 | 100 | 51.89 | 48.26 |
| | 7.33 | 0.4 | 100 | 42.78 | 47.76 |
| | 9.13 | 0.5 | 100 | 37.09 | 47.28 |
| | 3 | 0.2 | 110 | 80.89 | 45.28 |
| | 5.33 | 0.3 | 110 | 60.04 | 45.67 |
| | 7.33 | 0.4 | 110 | 56.59 | 46.12 |
| | 9.13 | 0.5 | 110 | 49.19 | 45.47 |
| | 3 | 0.2 | 120 | 83.22 | 44.69 |
| | 5.33 | 0.3 | 120 | 66.56 | 44.32 |
| | 7.33 | 0.4 | 120 | 53.83 | 43.9 |
| | 9.13 | 0.5 | 120 | 45.46 | 43.9 |
| E20b | 3 | 0.2 | 90 | 33.94 | 53.47 |
| | 5.33 | 0.3 | 90 | 22.29 | 52.29 |
| | 7.33 | 0.4 | 90 | 20.56 | 52.43 |
| | 9.13 | 0.5 | 90 | 19.25 | 50.66 |
| | 3 | 0.2 | 100 | 55.67 | 50.94 |
| | 5.33 | 0.3 | 100 | 45.97 | 49.99 |
| | 7.33 | 0.4 | 100 | 39.61 | 50.4 |
| | 9.13 | 0.5 | 100 | 34.28 | 50.6 |
| | 3 | 0.2 | 110 | 76.89 | 48.97 |
| | 5.33 | 0.3 | 110 | 59.7 | 49.4 |
| | 7.33 | 0.4 | 110 | 55.85 | 46.5 |
| | 9.13 | 0.5 | 110 | 53.08 | 46.44 |
| | 3 | 0.2 | 120 | 86.04 | 45.54 |
| | 5.33 | 0.3 | 120 | 67.61 | 45.07 |
| | 7.33 | 0.4 | 120 | 65.45 | 44.56 |
| | 9.13 | 0.5 | 120 | 55.87 | 44.81 |

### EXAMPLE 22

### The Temperature Programmed Oxidation Characterization of the Catalyst Before and after the Formaldehyde Treatment of this Patent

The surface of the catalyst with and without the formaldehyde treatment is performed by the use of temperature programmed oxidation (TPO). In the case of Nickel, each activated nickel atom can take up one oxygen atom as a result of oxidation and any adsorbed carbon atom may take up to a maximum of one oxygen atom for the formation of carbon monoxide or a maximum of two oxygen atoms for the formation of carbon dioxide. Each adsorbed carbon atom may take less than two oxygen atoms to form carbon dioxide if its adsorbed state involves bonds with oxygen. The same can also be said for the formation of carbon monoxide from its adsorbed precursor carbon species that involves bonds with oxygen.

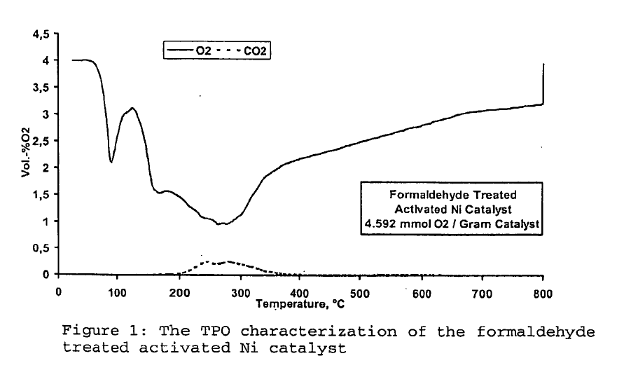

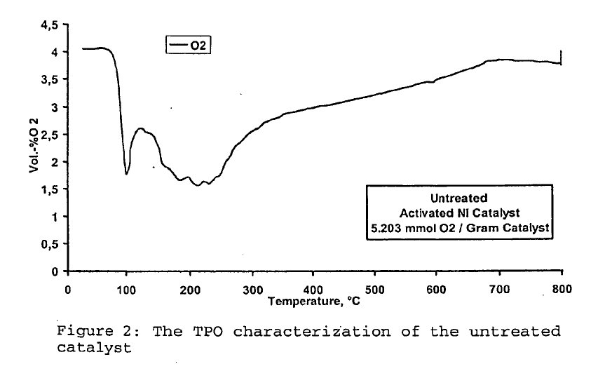

To perform TPO, about 5 to 10 grams of the water-moist catalyst is dried in a stream of nitrogen flowing 10 l/h at 120° C. for a period of 17 hours. The furnace is then carefully cooled to 20° C. After reaching a constant reactor temperature, the pure nitrogen is switched to a 4% oxygen in nitrogen mixture passing over the catalyst at the rate of 10 l/h while the temperature is ramped at the rate of 6° C./min to the end temperature of about 800° C. The oxygen content is measured during the experiment with a “Oxynos 100” paramagnetic detector and the amount of consumption is determined from the area of the oxygen curve. The CO₂ and CO contents are determined their specific detectors. In these experiments only CO₂ is detected. The formaldehyde treatment of the catalyst is performed per this invention as described by the previous examples via the aqueous treatment of the catalyst with formaldehyde without the use of an inert gas above the treatment solution, and this resulting catalyst is found to be very selective for the formation of primary amines via the hydrogenation of nitrites (i.e., benzonitrile). The results of these tests are in FIGS. 1 and 2. According to FIG. 1, the treated catalyst generates CO₂ during its TPO, indicating that its surface is covered with a carbon containing residue.

The comparison to FIG. 1 to FIG. 2 also shows, that the overall oxygen consumed per gram of catalyst is less for the formaldehyde treated catalyst in spite of the capability of the carbon residue to take up to a maximum of 2 oxygen atoms per adsorbed carbon atom. This shows that the treatment both covers the surface with carbon and modifies the total available surface area of the catalyst. One part of this modification is the oxidation of the surface during treatment via the oxygen present in the formaldehyde and/or the remaining dissolved oxygen in the treatment solution.

### EXAMPLE 23

### The Embedding of a Formaldehyde Treated Catalyst in a Primary Fatty Amine

Before embedding, the Raney-type Ni catalyst is treated with formaldehyde per this invention as described by the previous examples via the aqueous treatment of the catalyst with formaldehyde without the use of an inert gas above the treatment solution and this resulting catalyst is found to be very selective for the formation of primary amines via the hydrogenation of nitrites (i.e., benzonitrile). This treated catalyst is initially allowed to settle and the overstanding solution is then removed via suction. The remaining moist catalyst is heated under vacuum to remove as much as possible of the remaining moisture followed by the addition of the primary tallow amine, the homogenization of the mixture and the pastillation of this homogeneous mixture onto a cool surface to form embedded droplets of the modified catalyst in the primary amine. In comparison to the nonmodified catalyst, the catalyst of this invention generates far less ammonia and retains far more of the primary fatty amine during the embedding process and storage.

### EXAMPLE 24

### The Embedding of a Sodium Formate Treated Catalyst in a Primary Fatty Amine

A standard Raney-type Ni catalyst is initially allowed to settle and most of the overstanding solution is then removed via suction. Sodium formate is then given to the moist catalyst mass and stirred to homogeneity. The remaining moist homogeneous catalyst and sodium formate mixture is heated to 90° C. while continuing to stir for a period of one hour before applying a vacuum to remove as much as possible of the water followed by the addition of the primary tallow amine, the homogenization of the mixture and the pastillation of this homogeneous mixture onto a cool surface to form embedded droplets of the modified catalyst in the primary amine. In comparison to the nonmodified catalyst, the catalyst of this invention generates far less ammonia and retain far more of the primary fatty amine during the embedding process and storage.

1. A process for modifying a catalyst selected from the group consisting of Ni, Cu, Co and mixtures thereof by a modifier produced by the deposition of carbon containing residues with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in a liquid phase in the presence of one or more solvents as a catalyst treatment solution at temperatures ranging from above 0 to 150° C. for a period of time ranging from instant contact to longer than 24 hours, when the catalyst is to be stored in the catalyst treatment solution, characterized in that a gas phase over the catalyst treatment solution during treatment is air and no precautions are taken to remove the dissolved oxygen from the catalyst treatment solution,
or the gas phase over the catalyst treatment solution during treatment is an inert gas, when the catalyst is Cu catalysts,
or the gas phase over the catalyst treatment solution during the treatment is an inert gas or air, when the catalyst is selected from the group consisting of Ni, Cu, Co and mixtures thereof and the catalyst is doped or promoted with one or more elements,
or the gas phase over the catalyst treatment solution during treatment is an inert gas or air, when the catalyst is selected from the group consisting of Ni, Cu, Co and mixtures thereof and the catalyst is optionally doped or promoted with one or more elements and the liquid phase contains a templating agent,
or the gas phase over the catalyst treatment solution during the treatment is an inert gas or air, when the catalyst is selected from the group consisting of Ni, Cu, Co and mixtures thereof and the catalyst is optionally doped or promoted with one or more elements and/or the liquid phase contains a templating agent and/or one or more bases before, during and/or after addition of the modifier to the modification solution.
or the gas phase over the catalyst treatment solution during the treatment is an inert gas or air, when the catalyst is selected from the group consisting of Ni, Cu, Co and mixtures thereof and the catalyst is optionally doped or promoted with one or more elements and the liquid phase contains one or more bases before, during and/or after the addition of the modifier to the modification solution,
or the treatment of fixed bed catalysts, when the catalyst is selected from the group consisting of Ni, Cu, Co and mixtures thereof and the catalyst is optionally doped or promoted with one or more elements and the process is protected by an inert gas and/or a liquid phase present as either a thin film on the catalyst or a solution,
or the treatment of fixed bed catalysts, when the catalyst is selected from the group consisting of Ni, Cu, Co and mixtures thereof and the catalyst is optionally doped or promoted with one or more elements and/or in the presence of one or more bases and/or in the presence of a template where the process is protected by an inert gas and/or a liquid phase present as either a thin film on the catalyst or as a solution,
or the treatment of a catalyst selected from the group consisting of Ni, Cu, Co and mixtures thereof and the catalyst is optionally doped or promoted with one or more elements and optionally the modification medium contains a template and optionally the modification takes place in the presence of one or more bases, where the final modified catalyst is embedded in a fatty amine,
or the treatment of a catalyst selected from the group consisting of Ni, Cu, Co and mixtures thereof and the catalyst is optionally doped or promoted with one or more elements and optionally the modification medium contains a template and optionally the modification takes place in the presence of one or more bases, where the final catalyst is embedded in a primary fatty amine.

2. A process for modifying a catalyst selected from the group consisting of Ni, Cu, Co and mixtures thereof by the deposition of carbon containing residues with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in a liquid phase in the presence of one or more solvents to form a catalyst treatment solution at temperatures ranging from above 0 to 150° C. for a period of time ranging from instant contact to longer than 24 hours, when the catalyst is to be stored in the catalyst treatment solution, characterized in, that a gas phase over the catalyst treatment solution during treatment is air and no precautions were taken to remove the dissolved oxygen from the treatment solution.

3. A process for modifying a catalyst according to claim 1, where a catalyst selected from the group consisting of Ni, Cu, Co and mixtures thereof is modified by a modifier produced by the deposition of carbon containing residues with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in the liquid phase in the presence of one or more solvents at temperatures ranging from above 0 to 150° C., characterized by adding one or more bases before, during and/or after adding the modifier to the catalyst.

4. A process for modifying a catalyst according to claim 1, where a catalyst selected from the group consisting of Ni, Cu, Co and mixtures thereof is modified by a modifier produced by the deposition of carbon containing residues with formaldehyde via its contact with the catalyst in the liquid phase in the presence of one or more solvents to form a treatment solution at temperatures ranging from above 0 to 150° C. for a period of time ranging from instant contact to longer than 24 hours, when the catalyst is to be stored in the treatment solution, characterized in, that a gas phase over the catalyst treatment solution during treatment is air and no precautions were taken to remove the dissolve oxygen from the treatment solution.

5. A process for modifying a catalyst according to claim 1, characterized in, that a catalyst selected from the group consisting of Ni, Cu, Co and mixtures thereof is modified by the deposition of carbon containing residues with formaldehyde by its contact with the catalyst in a liquid phase in the presence of one or more solvents at temperatures ranging from above 0 to 150° C., where one or more bases is added to the catalyst before, during and/or after addition of formaldehyde.

6. A process for modifying a catalyst according to claim 1, characterized in, that a catalyst selected from the group consisting of Ni, Cu, Co and mixtures thereof is modified by the deposition of carbon containing residues with formaldehyde by its contact with the catalyst in a liquid phase in the presence of one or more solvents at temperatures ranging from above 0 to 150° C., where the bases NaOH, KOH and their mixtures are added to the catalyst before, during and/or after the addition of formaldehyde.

7. A process for modifying a catalyst according to claim 1, characterized in, that a catalyst selected from the group consisting of Ni, Cu, Co and mixtures thereof is modified by the deposition of carbon containing residues with metal salt formates by their contact with the catalyst in a liquid phase in the presence of one or more solvents at temperatures ranging from above 0 to 150° C.

8. A process for modifying a catalyst according to claim 1, characterized in, that a catalyst selected from the group consisting of Ni, Cu, Co and mixtures thereof is modified by the deposition of carbon containing residues with metal salt formates by their contact with the catalyst in a liquid phase in the presence of one or more solvents at temperatures ranging from above 0 to 150° C., and where one or more bases is added to the catalyst before, during and/or after the addition of sodium formate.

9. A process for modifying a catalyst according to claim 1, characterized in, that a catalyst selected from the group consisting of Ni, Cu, Co and mixtures thereof is modified by the deposition of carbon containing residues with metal salt formates by their contact with the catalyst in a liquid phase in the presence of one or more solvents at temperatures ranging from 70 to 130° C.

10. A process for modifying a catalyst according to claim 1, characterized in, that a catalyst selected from the group consisting of Ni, Cu, Co and mixtures thereof is modified by the deposition of carbon containing residues with metal salt formates by their contact with the catalyst in a liquid phase in the presence of one or more solvents at temperatures ranging from 70 to 130° C., and where one or more bases is added to the catalyst before, during and/or after the addition of sodium formate.

11. A process for modifying a catalyst according to claim 1, characterized in, that a Cu catalyst is modified via the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules, that interact strongly with metal surfaces via their contact with the catalyst in a liquid phase in the presence of one or more solvents as a treatment solution at temperatures ranging from above 0 to 150° C. for a period of time ranging from instant contact to longer than 24 hours, when the catalyst is to be stored in the treatment solution.

12. A process for modifying a catalyst according to claim 1, characterized in, that a Cu catalyst is modified by the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules, that interact strongly with metal surfaces by their contact with the catalyst in a liquid phase in the presence of one or more solvents at temperatures ranging from above 0 to 150° C., where one or more bases is added to the catalyst before, during and/or after the addition of the modifying agent.

13. A process for modifying a catalyst according to claim 1, characterized in, that a Cu catalyst is modified by the deposition of carbon containing residues with formaldehyde by its contact with the catalyst in a liquid phase in the presence of one or more solvents as the treatment solution at temperatures ranging from above 0 to 150° C. for a period of time ranging from instant contact to longer than 24 hours, when the catalyst is to be stored in the treatment solution.

14. A process for modifying a catalyst according to claim 1, where a Cu catalyst is modified by the deposition of carbon containing residues with formaldehyde by its contact with the catalyst in a liquid phase in the presence of one or more solvents at temperatures ranging from above 0 to 150° C., where one or more bases is added before, during and/or after the addition of formaldehyde.

15. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules, that interact strongly with metal surfaces via their contact with the catalyst in the liquid phase in the presence of one or more solvents as a treatment solution at temperatures ranging from above 0 to 150° C. for a period of time ranging from instant contact to longer than 24 hours, when the catalyst is to be stored in the treatment solution.

16. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by a modifier produced by the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules, that interact strongly with metal surfaces via their contact with the catalyst in the liquid phase in the presence of one or more solvents at temperatures ranging from above 0 to 150° C., where one or more bases is added to the catalyst before, during and/or after the addition of the modifier.

17. A process for modifying a catalyst according to claim 1, characterized in, that a catalyst selected from the group consisting of Ni, Cu, Co and mixtures thereof promoted with one or more of the elements selected from the periodic group consisting of 1A, 2A, IIIB, IVB, VB, VIIB, VIIB, VIII, IB, IIB, IIIA, IVA, VA, VIA and the rare earth elements is modified by the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules that interact strongly with metal surfaces via their contact with the catalyst in a liquid phase in the presence of one or more solvents as a treatment solution at temperatures ranging from above 0 to 150° C. for a period of time ranging from instant contact to longer than 24 hours, when the catalyst is to be stored in the treatment solution.

18. A process for modifying a catalyst according to claim 1, characterized in, that a catalyst selected from the group consisting of Ni, Cu, Co and mixtures thereof promoted with one or more of the elements selected from the periodic group consisting of 1A, 2A, IIIB, IVB, VB, VIIB, VIIB, VIII, IB, IIB, IIIA, IVA, VA, VIA and the rare earth elements are modified by the deposition of carbon containing residues with a material selected from the group consisting of formaldehyde, metal salt formates, carbon monoxide, carbon dioxide, aldehydes, ketones, amides, carboxylic acids, salts of carboxylic acids and other organic molecules, that interact strongly with metal surfaces via their contact with the catalyst in the liquid phase in the presence of one or more solvents at temperatures ranging from above 0 to 150° C., where one or more bases is added to the catalyst before, during and/or after the addition of the modifier.

19. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by the deposition of carbon containing residues with formaldehyde by its contact with the catalyst in a liquid phase in the presence of one or more solvents as a treatment solution at temperatures ranging from above 0 to 150° C. for a period of time ranging from instant contact to longer than 24 hours, when the catalyst is to be stored in the treatment solution.

20. A process for modifying a catalyst according to claim 1, characterized in, that a catalyst selected from the group consisting of Ni, Cu, Co and mixtures thereof promoted with one or more of the elements selected from the group consisting of 1A, 2A, IIIB, IVB, VB, VIIB, VIIB, VIII, IB, IIB, IIIA, IVA, VA, VIA and the rare earth elements are modified by the deposition of carbon containing residues with formaldehyde by its contact with the catalyst in a liquid phase in the presence of one or more solvents as a treatment solution at temperatures ranging from 5 to 130° C. for a period of time ranging from instant contact to longer than 24 hours, when the catalyst is to be stored in the treatment solution.

21. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by the deposition of carbon containing residues with formaldehyde by its contact with the catalyst in a liquid phase in the presence of one or more solvents as a treatment solution at temperatures ranging from above 0 to 150° C., where one or more bases is added to the catalyst before, during and/or after the addition of formaldehyde.

22. A process for modifying a catalyst according to claim 1, characterized in, that a catalyst selected from the group consisting of Ni, Cu, Co and mixtures thereof promoted with one or more of the elements selected from the group consisting of 1A, 2A, IIIB, IVB, VB, VIIB, VIIB, VIII, IB, IIB, IIIA, IVA, VA, VIA and the rare earth elements are modified by the deposition of carbon containing residues with formaldehyde by its contact with the catalyst in a liquid phase in the presence of one or more solvents as a treatment solution at temperatures ranging from above 0 to 150° C., where the addition of one or more bases is added to the catalyst before, during and/or after the addition of formaldehyde.

23. A process for modifying a catalyst according to claim 1, characterized in, that a catalyst selected from the group consisting of Ni, Cu, Co and mixtures thereof promoted with one or more of the elements elected from the group consisting of Mo, Cr, Fe, Re and V are modified by the deposition of carbon containing residues with sodium formate by its contact with the catalyst in a liquid phase in the presence of one or more solvents at temperatures ranging from above 0 to 150° C. for a period of time ranging from instant contact to longer than 24 hours, when the catalyst is to be stored in the treatment solution.

24. A process for modifying a catalyst according to claim 1, characterized in, that a catalyst selected from the group consisting of Ni, Cu, Co and mixtures thereof catalysts promoted with one or more of the elements selected from the group consisting of 1A, 2A, IIIB, IVB, VB, VIIB, VIIB, VIII, IB, IIB, IIIA, IVA, VA, VIA and the rare earth elements are modified by the deposition of carbon containing residues with sodium formate by its contact with the catalyst in a liquid phase in the presence of one or more solvents as a treatment solution at temperatures ranging from above 0 to 150° C. for a period of time ranging from instant contact to longer than 24 hours, when the catalyst is to be stored in the treatment solution.

25. A process for modifying a catalyst according to claim 1, characterized in, that a catalyst selected from the group consisting of Ni, Cu, Co and mixtures thereof promoted with one or more of the elements elected from the group consisting of Mo, Cr, Fe, Re and V are modified by the deposition of carbon containing residues with sodium formate by its contact with the catalyst in a liquid phase in the presence of one or more solvents at temperatures ranging from above 0 to 150° C., where of one of more bases is added to the catalyst before, during and/or after the addition of sodium formate.

26. A process for modifying a catalyst according to claim 1, characterized in, that a catalyst selected from the group consisting of Ni, Cu, Co and mixtures thereof promoted with one or more of the elements selected from the group consisting of 1A, 2A, IIIB, IVB, VB, VIIB, VIIB, VIII, IB, IIB, IIIA, IVA, VA, VIA and the rare earth elements are modified by the deposition of carbon containing residues with sodium formate by its contact with the catalyst in a liquid phase in the presence of one or more solvents at temperatures ranging from above 0 to 150° C., where one of more bases before, during and/or after the addition of sodium formate.

27. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by the deposition of carbon containing residues with sodium formate by its contact with the catalyst in a liquid phase in the presence of one or more solvents at temperatures ranging from 70 to 130° C.

28. A process for modifying a catalyst according to claim 1, characterized in, that a catalyst selected from the group consisting of Ni, Cu, Co and mixtures thereof promoted with one or more of the elements selected from the periodic group consisting of 1A, 2A, IIIB, IVB, VB, VIIB, VIIB, VIII, IB, IIB, IIIA, IVA, VA, VIA and the rare earth elements are modified by the deposition of carbon containing residues with sodium formate by its contact with the catalyst in a liquid phase in the presence of one or more solvents at temperatures ranging from 70 to 130° C.

29. A process for modifying a catalyst according to claim 1, characterized in, that a catalyst selected from the group consisting of Ni, Cu, Co and mixtures thereof promoted with one or more of the elements selected from the group consisting of Mo, Cr, Fe, Re and V are modified by the deposition of carbon containing residues with sodium formate by its contact with the catalyst in a liquid phase in the presence of one or more solvents at temperatures ranging from 70 to 130° C., where one or more bases is added before, during and/or after the addition of sodium formate.

30. A process for modifying a catalyst according to claim 1, characterized in, that a catalyst selected from the group consisting of Ni, Cu, Co and mixtures thereof promoted with one or more of the elements selected from the group consisting of 1A, 2A, IIIB, IVB, VB, VIIB, VIIB, VIII, IB, IIB, IIIA, IVA, VA, VIA and the rare earth elements are modified by the deposition of carbon containing residues with sodium formate by its contact with the catalyst in a liquid phase in the presence of one or more solvents at temperatures ranging from 70 to 130° C., where one or more bases is added to the catalyst before, during and/or after the addition of sodium formate.

31. A process for modifying a catalyst according to claim 1, characterized in, that a catalyst selected from the group consisting of Ni, Cu, Co and mixtures thereof as fixed bed catalysts are modified in a liquid phase.

32. A process for modifying a catalyst according to claim 1, characterized in, that a catalyst selected from the group consisting of Ni, Cu, Co and mixtures thereof as fixed bed catalysts are modified in a liquid phase, except that modification is carried out in a flooded fixed bed reactor either with or without recirculation.

33. A process for modifying a catalyst according to claim 1, characterized in, that a catalyst selected from the group consisting of Ni, Cu, Co and mixtures thereof as fixed bed catalysts are modified in a liquid phase, except that the modification is performed in a trickle phase in a fixed bed reactor and that an atmosphere surrounding the catalyst is either an inert atmosphere or a reducing atmosphere.

34. A process for modifying a catalyst according to claim 1, characterized in, that a catalyst selected from the group consisting of Ni, Cu, Co and mixtures thereof as fixed bed catalysts are modified in a liquid phase, except that the modification is performed with an aerosol of the modifier in a fixed bed reactor and that an atmosphere surrounding the catalyst is either an inert atmosphere or a reducing atmosphere.

35. A process for modifying a catalyst according to claim 1, characterized in, that a catalyst selected from the group consisting of Ni, Cu, Co and mixtures thereof as fixed bed catalysts are modified in a liquid phase, except that the modification is performed in a gas phase with the modifier that is able to exist in the gas phase under the modifying conditions in a fixed bed reactor and that an atmosphere surrounding the catalyst is either an inert atmosphere or a reducing atmosphere.

36. A process for modifying a catalyst according to claim 1, characterized in, that a catalyst selected from the group consisting of Ni, Cu, Co and mixtures thereof as fixed bed catalysts are modified in a liquid phase, except that the modification is performed in a gas phase with the modifiers a modifier that is able to exist in the gas phase under the modifying conditions in a fixed bed reactor, where the treatment temperature can range from −50 to 500° C. and that an atmosphere surrounding the catalyst is either an inert atmosphere or a reducing atmosphere.

37. A process according to claim 31, characterized in, that a catalyst selected from the group consisting of Ni, Cu, Co and mixtures thereof as fixed bed catalysts are modified, except that the modification temperature can range from above 0 to 150° C.

38. A process for modifying a catalyst according to claim 1, characterized in, that the modified Ni, Cu, Co and mixtures thereof catalyst is embedded in a primary fatty amine.

39. A process for modifying a catalyst according to claim 1, characterized in, that the modified Ni, Cu, Co and mixtures thereof catalyst is embedded in a fatty amine.

40. A process for modifying a catalyst according to claim 1, characterized in, that catalysts are modified, where catalytic metals are selected from the group consisting of Fe, Pd, Pt, Ru, Ag, Au, V, Re, W, Mo, Rh, Ir and mixtures thereof.

41. A process for modifying a catalyst according to claim 1, characterized in, that catalysts are modified, where modification is performed in the presence of a templating agent.

42. A process for modifying a catalyst according to claim 1, characterized in, that the carbon depositing modification is performed under reducing atmosphere.

43. Process for the improved stereoselective hydrogenation of compounds characterized in, that a catalyst modified by the process according to claim 1 is used.

44. Process for the improved stereoselective hydrogenation of fructose to generate a higher concentration of mannitol characterized in, that a catalyst modified by the process according to claim 1 is used.

45. Process for the improved chemoselective hydrogenation of compounds characterized in, that a catalyst modified by the process according to claim 1 is used.

46. Process for the improved chemoselective hydrogenation of compounds where the more strongly adsorbed moeity is hydrogenated over the less strongly adsorbed one characterized in, that a catalyst modified by the process according to claim 1 is used.

47. Process for the improved chemoselective hydrogenation of compounds where nitrites are preferentially hydrogenated over olefins characterized in, that a catalyst modified by the process according to claim 1 is used.

48. Process for the improved chemoselective hydrogenation of compounds where carbonyls are preferentially hydrogenated over olefins characterized in, that a catalyst modified by the process according to claim 1 is used.

49. Process for the improved chemoselective hydrogenation of compounds, where unsaturated fatty amines are produced from unsaturated fatty nitrites, characterized in, that a catalyst modified by the process according to claims 1, 38 and 39 claim 1 is used.

50. Process for the improved regioselective hydrogenation of compounds, characterized in, that a catalyst modified by the process according to claim 1 is used.

51. Process for the improved regioselective hydrogenation of compounds, where the less sterically hindered moeity is preferentially hydrogenated over the more sterically hindered one characterized in, that a catalyst modified by the process according to claim 1 is used.

52. Process for the improved regioselective hydrogenation of compounds, where exocyclic moieities are preferentially hydrogenated over endocyclic ones, characterized in, that a catalyst modified by the process according to claim 1 is used.

53. Process for the improved regioselective hydrogenation of compounds, where terminal moeities are preferentially hydrogenated over internal ones, characterized in, that a catalyst modified by the process according to claim 1 is used.

54. Process for the improved regioselective hydrogenation of compounds, where moeities with fewer and less bulkier groups attached to them are preferentially hydrogenated over moeities with more and/or bulkier groups attached to them, characterized in, that a catalyst modified by the process according to claim 1 is used.

55. Process for the improved hydrogenation of nitrites to primary amines, characterized in, that a catalyst modified by the process according to claim 1 is used.

56. Process for the improved conversion of nitrites to the corresponding dimerized disubstituted imines, characterized in, that a catalyst modified by the process according to claim 1 is used.

57. Process for the improved hydrogenation of nitrites to primary amines in the presence of ammonia, characterized in, that a catalyst modified by the process according to claim 1 is used.

58. Process for the improved hydrogenation of nitrites to primary amines in the presence of one or more bases, characterized in, that a catalyst modified by the process according to claim 1 is used.

59. Process for the improved hydrogenation of nitrites to primary amines in the presence of NaOH, KOH or their mixtures, characterized in, that a catalyst modified by the process according to claim 1 is used.

60. Process for the improved hydrogenation of aromatic nitrites to primary amines, characterized in, that a catalyst modified by the process according to claim 1 is used.

61. Process for the improved hydrogenation of aromatic nitrites to primary amines in the presence of ammonia, characterized in, that a catalyst modified by the process according to claim 1 is used.

62. Process for the improved hydrogenation of aromatic nitrites to primary amines in the presence of bases, characterized in, that a catalyst modified by the process according to claim 1 is used.

63. Process for the improved hydrogenation of alpha, omega dinitriles to amino nitrites, characterized in, that a catalyst modified by the process according to claim 1 is used.

64. Process for the improved hydrogenation of nitro groups without the formation of dimers, characterized in, that a catalyst modified by the process according to claim 1 is used.

65. Process for the improved hydrogenation of compounds, where the performance of the catalyst is fitted to the capabilities of the reactor, characterized in, that a catalyst modified by the process according to claim 1 is used.

66. Process for the improved hydrogenation of triglycerides to desired iodine values, characterized in, that a catalyst modified by the process according to claim 1 is used.

67. Process according to claim 43, characterized in, that modified catalysts are used either in a liquid phase, a gas phase, a trickle phase or in an aerosol of the reactant(s) by either a single pass or a recycling process in either a stirred tank reactor, a fluidized bed reactor or a fixed bed reactor as determined by the best fit to the reaction type and catalyst technology.

68. Process according to claim 43, characterized in, that the catalysts modified with catalytic metals selected from the group consisting of Fe, Pd, Pt, Ru, Ag, Au, V, Re, W, Mo, Rh, Ir and mixtures thereof either in liquid phase, gas phase, trickle phase or in an aerosol of the reactant(s) by either a single pass or a recycling process in either a stirred tank reactor, a fluidized bed reactor or a fixed bed reactor.
